(19) United States
(12) Patent Application Publication
(10) Pub. No.: US 2021/0087631 A1
(43) Pub. Date: Mar. 25, 2021

(54) METHODS FOR ASSESSING RISK OF OR DIAGNOSING GENETIC DEFECTS BY IDENTIFYING DE NOVO MUTATIONS OR SOMATIC MOSAIC VARIANTS IN SPERM OR SOMATIC TISSUES

(71) Applicant: The Regents of the University of California, Oakland, CA (US)

(72) Inventors: Joseph GLEESON, La Jolla, CA (US); Martin BREUSS, La Jolla, CA (US); Kiely JAMES, La Jolla, CA (US); Jonathan SEBAT, La Jolla, CA (US); Morgan KLEIBER, La Jolla, CA (US); Danny ANTAKI, La Jolla, CA (US); William BRANDLER, La Jolla, CA (US)

(21) Appl. No.: 16/499,105

(22) PCT Filed: Mar. 28, 2018
(86) PCT No.: PCT/US2018/024878

§ 371 Date: Sep. 27, 2019

Related U.S. Application Data
(60) Provisional application No. 62/478,005, filed on Mar. 28, 2017.
(60) Provisional application No. 62/512,368, filed on May 30, 2017.

(51) Int. Cl. C12Q 1/6883; G16B 20/20

(52) U.S. Cl. CPC C12Q 1/6883; G16B 20/20; C12Q 2600/172; C12Q 2600/158; C12Q 2600/156

(57) ABSTRACT

Provided are compositions and methods for assessing the genetic makeup of sperm comprising use of Digital Droplet PCR, wherein optionally the genetic makeup of the sperm is screened for the presence of a genetic defect or trait, or the genetic makeup of the sperm is screened for a de novo genetic mutation. Provided are compositions, including products of manufacture and kits, and methods, for determining the risk of inheritance of a genetic defect or trait in a younger child or a potential sibling, wherein the younger child or potential sibling has an older sibling having the genetic defect or trait. Provided are compositions and methods for determining a man or woman's risk of having a child with a genetic defect or a disease caused by a genetic defect or a trait such as autism, schizophrenia, heart disease, congenital heart disease or a neurocutaneous disease.

### RELATED APPLICATIONS

This application is a national phase application claiming benefit of priority under 35 U.S.C. § 371 to Patent Convention Treaty (PCT) International Application serial number PCT/US2018/024878, filed Mar. 28, 2018, now pending, which claims the benefit of priority to U.S. Provisional Application No. 62/478,005 filed Mar. 28, 2017 and U.S. Provisional Application No. 62/512,368, filed May 30, 2017. The aforementioned applications are expressly incorporated herein by reference in their entirety and for all purposes.

### STATEMENT AS TO FEDERALLY SPONSORED RESEARCH

This invention was made with government support under MH076431 awarded by the National Institutes of Health. The government has certain rights in the invention.

### TECHNICAL FIELD

This invention generally relates to human genetics and the diagnosis and treatment of hereditable genetic traits. In alternative embodiments, provided are compositions, including products of manufacture and kits, and methods, for assessing the genetic makeup of sperm comprising use of Digital Droplet PCR (ddPCR), wherein optionally the genetic makeup of the sperm is screened for the presence of a genetic defect or trait, and optionally the genetic makeup of the sperm is screened for a de novo genetic mutation. In alternative embodiments, provided are compositions, including products of manufacture and kits, and methods, for determining the risk of inheritance of a genetic defect or trait in a younger child or a potential sibling, wherein the younger child or potential sibling has an older sibling having the genetic defect or trait. In alternative embodiments, provided are methods for determining the risk of inheritance of a genetic defect or trait, or a haploinsufficient disease or trait, in a younger child or a potential sibling wherein optionally the haploinsufficient disease or trait is autism, schizophrenia, heart disease, congenital heart disease or a neurocutaneous disease.

In alternative embodiments provided are compositions and methods for detecting and quantifying the presence of a disease- or trait-causing mutation in a sample of sperm, blood, saliva, buccal cells or other tissue from a prospective father and/or from blood, saliva, buccal cells or other tissue from a prospective mother, thereby providing to the parents an estimate of their risk of transmitting a disease- or trait-causing mutation to an offspring. In alternative embodiments, provided are compositions, including products of manufacture and kits, and methods, for determining the risk of inheritance of a genetic defect or trait in a younger child or a potential sibling, wherein the younger child or potential sibling to be assessed for inheritance of the genetic defect or trait has a sibling already diagnosed with that genetic defect or trait. In alternative embodiments, the methods are applied as a “Non-Invasive Prenatal Test” (NIPT) for detecting the presence of mutations in fetal nucleic acid (e.g., DNA) that is circulating in the blood of a pregnant mother. In alternative embodiments, the trait, disease or condition caused by the genetic defect is autism, schizophrenia, heart disease, congenital heart disease or a neurocutaneous disease.

### BACKGROUND

In general, the risk of having a child with autism spectrum disorder (ASD) is about 1 in 68, or 1.5%. But the risk goes up for families who already have a child with ASD. If a family has one child with ASD, the chance of the next child having ASD is about 20%. If the next child is a boy, the risk is 26%, whereas if it is a girl the risk is 10%. About 4-7% of families had more than one child with autism. Since most people with autism do not reproduce, most of this risk is thought to be due to germline mosaicism.

Currently if a child has a birth defect or autism, the emerging trend is to perform whole exome sequencing to identify de novo genetic mutations. These mutations overwhelmingly come from the father, because sperm cells but not egg cells continue to divide through the life of adults. Once the mutation is identified, the diagnosis can be made in the child, but the parents are left wondering if this genetic event could recur in future children.

Males produce 1500 sperm cells per second throughout life, and most of these individual cells are thought to derive from a collection of perhaps a few thousand sperm stem cells. Thus, by assessing a collection of thousands of sperm, the sensitivity of the assay to assess for mutation is very high. The sensitivity is not 100% though, and there is still the possibility that a single sperm carries a genetic mutation that can cause disease.

There have been numerous reports attributing de novo mutations as a cause for birth defects such as congenital heart disease, neurocutaneous disorders, autism and schizophrenia [1-5]. Risk of recurrence in families is in the range of 10%. For instance, germline mosaicism was detected in 11.6% of parents of children with Duchenne/Becker muscular dystrophy [6].

Recent studies have begun to address somatic rather than germline mosaicism. For instance, in one study of 100 families with de novo mutation, there was evidence of somatic mosaicism in one of the parents in 4 cases assessed from blood [7]. However, somatic mosaicism rates are very rare for transmitted mutations, and thus not useful to determine personal risk at scale. There are also studies that perform exome sequencing on the fetus when found to have a structural defect based upon ultrasound, where between 10% to 27% of cases had likely genetic diagnosis made prior to birth [8]. However, families want this information prior to conceiving a fetus with a genetic mutation.

There have been reports in the literature of an increased risk of psychiatric disorders such as autism and schizophrenia with increased age of the father at the time of conception [9-15]. There are papers that propose mechanisms by which de novo mutations in sperm lead to over-proliferation of specific clones, which has been proven in only a few examples [16], which could be one mechanism by which age influences the rate of de novo sperm mutations. Most but not all risk is thought to result from an age-dependent effect on the accumulation of de novo mutations, whereas some of the increased risk of autism in older fathers due to de novo mutations was postulated to be from age-independent effects, which remains an active area of research [17].

Currently there is no genetic assessment of sperm available commercially, and no publications on the application of using sperm as a way to assess risk of childhood disease. Currently there is no risk assessment available for couples that have had a child with a genetic disease due to de novo genetic mutation. Currently the only non-invasive prenatal test that is commercially available is one for detecting a small number of extra chromosomes that can form viable offspring (Trisomy 21, 18, 13). No NIPT test currently available can detect single nucleotide variants (SNVs) or structural variants (SVs).

### SUMMARY

### Methods for Assessing Risk of or Diagnosing Genetic Defects in Children by Identifying De Novo Mutations in Male Sperm

In alternative embodiments, provided are compositions (e.g., kits) and methods for assessing the genetic makeup of sperm comprising use of a ‘haploinsufficiency-ome’, and optionally using a Digital Droplet PCR (ddPCR) to sequence the genetic makeup of the sperm, wherein the methods or compositions comprise, or comprise use of:
(a) providing a sperm or sperm sample, or sample of the genome of a sperm or sperm sample;
(b) providing a ‘haploinsufficiency-ome’ database, or a compilation of gene sequences, of a comparable species or animal (e.g., optionally providing a human ‘haploinsufficiency-ome’ to compare with a human sperm sample), wherein the ‘haploinsufficiency-ome’ comprises a database or compilation of gene sequences from sperm or haploid precursors thereof;
(c) sequencing the sperm's genome, or the sperm's DNA, and optionally the sequencing comprises using a method comprising a Digital Droplet polymerase chain reaction (PCR) (ddPCR, digital PCR or dePCR), or equivalent (optionally a QX200™ or AutoDG™ Droplet Digital™ PCR System (BIO-RAD)); and
(d) comparing the sequenced sperm genome or DNA with the ‘haploinsufficiency-ome’ database or compilation of gene sequences, and determining any sequence differences,

wherein optionally the ‘haploinsufficiency-ome’ is a “disease-ome” (a panel of genes that produce or are associated with haploinsufficient birth defects or other diseases wherein one copy of a gene is defective, mutated or missing) or a “hereditable condition-ome” (a panel of genes that produce or are associated with a hereditable condition or trait, wherein one copy of a gene is defective, mutated or missing),

and optionally the “disease-ome” or “hereditable condition-ome” is:
an ‘autism-ome’ (a panel of genes that produce or are associated with autism (or autism spectrum disorder (ASD)), wherein one copy of the gene is defective, mutated or missing), or having a specific mutation or allele associated with autism or ASD,
a schizophrenia-ome' (a panel of genes that produce or are associated with schizophrenia, wherein one copy of the gene is defective, mutated or missing), or having a specific mutation or allele associated with schizophrenia,
a ‘congenital heart disease-ome’ (a panel of genes that produce or are associated with congenital heart disease, wherein one copy of the gene is defective, mutated or missing), or having a specific mutation or allele associated with congenital heart disease,
a spina bifida-ome (a panel of genes that produce or are associated with spina bifida, wherein one copy of the gene is defective, mutated or missing), or having a specific mutation or allele associated with spina bifida, or
a compilation of gene sequences of any disease class or hereditable condition or trait class where one or more de novo mutations are known to contribute (optionally substantially) to risk of a child acquiring (inheriting) the disease or hereditable condition or trait,

and optionally the genetic makeup of the sperm is screened for the presence of a genetic defect, hereditable condition or trait, wherein a finding or a determination of one or more sequence differences in step (d) in the sperm sample versus the “disease-ome” or “hereditable condition-ome” is a finding or determination that a progeny of the sperm is at risk, optionally at high risk, of developing or inheriting the disease, condition or trait (if the screened sperm's genetic makeup comprises one or more sequences or sequence variants that specifically matches a “disease-ome” or “hereditable condition-ome” sequence, this is a finding or determination that a progeny of the sperm is at risk, optionally at high risk, of developing or inheriting the disease, condition or trait_{[A1]}),

wherein optionally when the progeny of the spelin is at risk of developing or inheriting the disease, condition or trait, the progeny of the sperm has a greater than 1%, 3%, 4%, 5%, 6%0, 7%, 8%, 9%0, or 10% greater chance of developing or inheriting the disease, condition or trait than when a sperm does not have one or more sequences or sequence variants that specifically matches a “disease-ome” or “hereditable condition-ome” sequence,

wherein optionally when the progeny of the sperm is at high risk of developing or inheriting the disease, condition or trait, the progeny of the sperm has a greater than 11%, 12%, 13%, 14%, 15%, 16%, 17%, 18%, 19%, or 20% or more greater chance of developing or inheriting the disease, condition or trait than when a sperm does not have one or more sequences or sequence variants that specifically matches a “disease-ome” or “hereclitable condition-ome” sequence, _{[A2]}

and optionally the genetic makeup of the sperm is screened for a de novo genetic mutation, or the genetic defect or trait comprises a de novo genetic mutation, and optionally if the one or more sequence differences in step (d) in the sperm sample versus the “disease-ome” or “hereditable condition-ome” is a finding or determination that the sperm has a de novo genetic mutation, then this is a finding or determination that a progeny of the sperm is at risk, optionally at high risk, of inheriting the de novo genetic mutation,

wherein optionally the sperm is a human or a non-human sperm.

In alternative embodiments, provided are methods for determining the risk of inheritance of a genetic defect or trait in a younger child or a potential sibling, wherein the younger child or potential sibling has an older sibling having the genetic defect or trait, comprising:

determining the genetic makeup of the sperm of the father of the older sibling using a method known in the art or as provided herein, and determining whether the genetic makeup of the sperm has the genetic defect or trait found in the older sibling;

wherein determining that the sperm of the father has the genetic defect or trait found in the older sibling indicates a risk_{[A3]} that the younger child or the potential sibling will inherit the genetic defect or trait found in the older sibling, or that the genetic defect or trait found in the older sibling will be transmitted to the younger child or the potential sibling.

In alternative embodiments of the methods, the older sibling has autism or autism spectrum disorder (ASD), and a genetic defect or trait found in an ‘autism-ome’ is detected in the sperm of the father, or a specific mutation or allele associated with autism or autism spectrum disorder (ASD) is detected in the sperm of the father, thereby detecting in increased risk_{[A4]} of autism or autism spectrum disorder (ASD) in the younger child or the potential sibling.

In alternative embodiments, provided are methods for determining the risk of inheritance of a genetic defect or trait, or a haploinsufficient disease or trait, in a younger child or a potential sibling, comprising:

determining the genetic makeup of the sperm of the father using a method known in the art or as provided herein, and determining whether the genetic makeup of the sperm comprises a genetic defect or trait, or a haploinsufficient disease or trait, wherein optionally the genetic defect or trait is a de novo genetic defect or trait, and optionally the genetic defect or trait is a genetic defect or trait found in a ‘haploinsufficiency-ome’, or an ‘autism-ome’, or a disease or trait associated with a specific mutation or allele,

wherein determining that the sperm of the father has the genetic defect or trait found indicates a risk_{[A5]} that the younger child or the potential sibling will inherit the genetic defect or trait, or that the detected genetic defect or trait will be transmitted to the younger child or the potential sibling,

and optionally the haploinsufficient disease or trait is an autism or autism spectrum disorder (ASD), a trinucleotide expansion, an intellectual disability, a schizophrenia, a heart disease, a congenital heart disease, a neurocutaneous disease, a chromosomal rearrangement, a cancer, dyskeratosis congenita (DKC), Marfan syndrome (MFS) or cleidocranial dysostosis (CCD).

In alternative embodiments, provided are methods for determining the risk that a child or potential child has or will have autism or autism spectrum disorder (ASD), comprising:

determining the genetic makeup of the sperm of the father using a method as provided herein, and determining whether the genetic makeup of the sperm comprises a genetic defect or trait found in an ‘autism-ome’, or a specific mutation or allele associated with the genetic defect or trait, wherein optionally the genetic defect or trait is a de novo genetic defect or trait,

wherein determining that the sperm of the father has the ‘autism-ome’ or specific genetic defect or trait found indicates a risk_{[A6]} that the younger child or the potential sibling will inherit autism or autism spectrum disorder (ASD), or that autism or autism spectrum disorder (ASD) will be transmitted to the younger child or the potential sibling.

In alternative embodiments, provided are kits or products of manufacture comprising components for practicing the method of any of the preceding claims, or a method as provided herein, wherein optionally the kit or the product of manufacture comprises PCT primers for detecting the desired genetic defect or trait, and optionally the kit or the product of manufacture comprises instructions for practicing the method of any of the preceding claims, or a method as provided herein.

In alternative embodiments, provided are Uses of the product of manufacture or a kit as provided herein, for determining the risk of inheritance of a genetic defect or trait in a younger child or a potential sibling, or determining the risk that a child or potential child has or will have autism.

### Methods for Inferring Disease Risk in Offspring by Detection of Somatic Mosaic Variants in Parental Sperm or Somatic Tissues

In alternative embodiments, provided are compositions (e.g., kits) and methods for determining the presence of a genetic or DNA variation in a sample from an individual,

wherein the genetic or DNA variation comprises: a Structural Variant (SV), a single nucleotide variant (SNV), or an indel (comprising mutations resulting in either insertion or deletion, or both insertion and deletion, of bases in DNA),

the method comprising:

(a)
(i) providing:
   a tissue, fluid, blood, serum, sperm or sperm sample, or a sample of the genome of or a genome derived from the tissue, fluid, blood, serum, sperm or sperm sample, or
   DNA from or DNA derived from a tissue, fluid, blood, serum, sperm or sperm sample;
(ii) detecting a variation or a mutation in a DNA from (or in) the sample, or detecting a variation or a mutation in the sequence of the DNA from or in) the sample,
wherein optionally the DNA is analyzed (and the variation or the mutation in the DNA is detected, or the sequence of the DNA is determined) by a method comprising use of:
   (1) breakpoint polymerase chain reaction (PCR) to detect a DNA breakpoint comprising use of a set of nested primers that span the junction of a structural variant (SV), wherein optionally the presence of the DNA breakpoint can be detected at frequencies <1%;
   (2) digital droplet PCR (ddPCR) or an emulsion PCR method to quantify mutations at the level of individual chromosomes;
   (3) restriction site mutation (RSM) detection comprising use of a set of nested primers that span a single-nucleotide variant, wherein a mutation can be detected by first eliminating the reference sequence by digestion with a restriction enzyme followed by amplification of the mutant sequence by serial PCR reactions using nested primers;
   (4) any combination of (1) and (2), (1) and (3), (2) and (3), or (1), (2) and (3); or
   (5) whole genome sequencing; or

(b) detecting a variation or a mutation in a DNA from (or in) a sample, or detecting a variation or a mutation in the sequence of the DNA from (or in) a sample,

wherein the sample comprises
a tissue, fluid, blood, serum, sperm or sperm sample, or a sample of the genome of or a genome derived from the tissue, fluid, blood, serum, sperm or sperm sample, or
DNA from or DNA derived from a tissue, fluid, blood, serum, sperm or sperm sample;

and optionally the DNA is analyzed, or the sequence of the DNA is determined, by a method comprising use of:
(1) breakpoint polymerase chain reaction (PCR) to detect a DNA breakpoint comprising use of a set of nested primers that span the junction of a structural variant (SV), wherein optionally the presence of the DNA breakpoint can be detected at frequencies <1%;
(2) digital droplet PCR (ddPCR) or an emulsion PCR method to quantify mutations at the level of individual chromosomes;
(3) restriction site mutation (RSM) detection comprising use of a set of nested primers that span a single-nucleotide variant, wherein a mutation can be detected by first eliminating the reference sequence by digestion with a restriction enzyme followed by amplification of the mutant sequence by serial PCR reactions using nested primers;
(4) any combination of (1) and (2), (1) and (3), (2) and (3), or (1), (2) and (3); or
(5) whole genome sequencing.

In alternative embodiments, the methods further comprise quantifying a mutation frequency of the DNA variation or a mutation to provide an estimate of the risk_{[A7]} of the presence or possible occurrence of a disease, trait or disorder caused by the genetic mutation or variation in an offspring or a potential future child.

In alternative embodiments, the methods are used as a Non-Invasive Prenatal Test (NIPT) when the father is known to carry a mutation in his sperm and the same mutation is undetectable in the blood of the mother prior to her pregnancy, wherein detection of the DNA variation or mutation in the mother's blood, serum or plasma, during pregnancy determines the presence or occurrence of the genetic mutation in the fetus, and thereby also provides an estimate of the risk_{[A8]} of the presence or possible occurrence of a disease, trait or disorder caused by the genetic mutation or variation in the child or fetus.

In alternative embodiments of the methods, an older sibling has autism or autism spectrum disorder (ASD), and a genetic defect or trait is detected in the DNA of the sperm of the father, or a specific mutation or allele associated with autism or autism spectrum disorder (ASD) is detected in the sperm of the father, thereby detecting an increased risk_{[A9]} of autism or autism spectrum disorder (ASD) in the younger child or the potential sibling.

In alternative embodiments, the disease, trait or disorder is a haploinsufficient or dominant disease or trait; or the disease, trait or disorder is: an autism or autism spectrum disorder (ASD), a trinucleotide expansion, an intellectual disability, a schizophrenia, a heart disease, a congenital heart disease, a neurocutaneous disease, a chromosomal rearrangement, a cancer, dyskeratosis congenita (DKC), Marfan syndrome (MFS) or cleidocranial dysostosis (CCD).

In alternative embodiments, provided are kits or products of manufacture comprising components for practicing a method as provided herein, wherein optionally the kit or the product of manufacture comprises PCR primers for detecting a desired genetic defect, disease or trait, and optionally the kit or the product of manufacture comprises instructions for practicing the method of any of the preceding claims.

In alternative embodiments, provided are Uses of the product of manufacture or the kit as provided herein, for determining the risk of inheritance of a genetically-inherited disease, trait or disorder in a younger child or a potential sibling (future offspring), or determining the risk that a child or potential child has or will have a genetically-inherited disease, trait or disorder, and optionally the disease, trait or disorder comprises or is: an autism or autism spectrum disorder (ASD), a trinucleotide expansion, an intellectual disability, a schizophrenia, a heart disease, a congenital heart disease, a neurocutaneous disease, a chromosomal rearrangement, a cancer, dyskeratosis congenita (DKC), Marfan syndrome (MFS) or cleidocranial dysostosis (CCD).

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A-B schematically illustrates an exemplary protocol comprising steps involved in genetic profiling of sperm. FIG. 1A: Ejaculate containing sperm are evaluated under microscope then centrifuged in isotonic solution to pellet sperm cells, then washed and lysed and disrupted with steel beads to collect DNA using column purification followed by concentration assessment. FIG. 1B: Purified DNA from male sperm compared with blood or saliva sample, is used to perform either unbiased whole exome sequencing (WES), whole genome sequencing (WGS) (top), to perform candidate sanger sequencing if necessary (middle), or to perform ddPCR (bottom). The results from ddPCR show digital droplets of several types. Droplets in black contained no DNA and are discarded. Droplets in green contain mutant DNA. Droplets in red contain normal DNA. Droplets in orange (double positive) contain both normal and mutant DNA. Counting the number of droplets of each color provides quantitative measurement of the level of somatic mosaicism.

FIG. 2A-B graphically illustrates data confirming a germline mosaicism assessed from father's sperm. FIG. 2A: De novo mutation was confirmed from Sanger sequencing of a father and affected from saliva as a C to T mutation. Note that father's saliva contains no evidence of a mutant peak, whereas affected's saliva shows peaks of equal height of C and T (equal height of red and blue) meaning that the affected is heterozygous for the mutation. Father's sperm sample contains evidence of a minor peak (red) under the blue peak, calculated that about 15% mosaicism. However most germline mosaicism is not detectable if less than about 10% using Sanger sequencing. FIG. 2B: Relative abundance of mutation (%) from ddPCR. Affected saliva sample showed 46.8% mutant, mother saliva showed <0.1% mutant, and father's saliva sample showed 1.2% mosaicism. Control blood and sperm sample from healthy donor showed no evidence of mutation. Father's sperm sample showed 14.9% mosaicism. Thus the results from the sperm testing indicates an enrichment for mosaicism in father's sperm, and conveys a 14.9% chance that future children will inherit a sperm with this mutation.

FIG. 3A-D graphically illustrates data from a ddPCR analysis of saliva and sperm from same family above: FIG. 3A is paternal saliva, 1.2%; FIG. 3B is material saliva, less than 0.1%; FIG. 3C is affected saliva, 46.8%; and FIG. 3D is paternal sperm, 14.9%. For all FIG. 3A-D: blue dots (top left quadrant) indicate mutant droplets, green dots (bottom right) indicate wildtype droplets, orange dots (top right) indicate droplets with both mutant and wildtype copy of DNA, black droplets (bottom left) indicate droplets without DNA. Counting the number of droplets of each color provides quantitative measurement of the level of somatic mosaicism in each tissue assessed.

FIG. 4 graphically illustrates the percent mosaicism, or the allelic fraction, as a function of the number of variants, as described in Example 4, below; the figure is an example of detection of mosaicism from sperm assessment.

FIG. 5A-B schematically and graphically illustrates detection of a somatic mosaic Structural Variant in paternal sperm and blood by nested PCR. FIG. 5A schematically illustrates: a de novo deletion of the gene CACNG2 as originally detected by 30× whole genome sequencing blood-derived DNA; the gene, as depicted by the red band, is 128,195 base pairs (bp) in length, as discussed in further detail, below.

FIG. 6A-B shows a digital droplet PCR detection and quantification of a somatic mosaic Structural Variant in paternal sperm and blood, and in particular, graphically illustrates data quantifying the number of copies of the CACNG2 deletion allele that are present in paternal sperm and blood in REACH family F0001; FIG. 6A graphically illustrates fluorescence amplitude versus various fractions of material and maternal blood, and sperm; FIG. 6B graphically illustrates copy number of the CACNG2 deletion in these samples, as discussed in further detail, below.

FIG. 7 graphically illustrates data from the detection and quantification of a somatic mosaic Structural Variant by whole genome sequencing; and in particular, shows the proportion of structural variant (SV) supporting reads in various samples of material and paternal blood, and sperm, as discussed in further detail, below.

Like reference symbols in the various drawings indicate like elements.

### DETAILED DESCRIPTION

### Methods for Assessing Risk of or Diagnosing Genetic Defects in Children by Identifying De Novo Mutations in Male Sperm

In alternative embodiments, provided are compositions, including products of manufacture and kits, and methods, for analyzing the genetic content of male sperm to assess whether the sperm carries de novo mutations coming from the father. In alternative embodiments, this method can be used to assess risk of a couple who has a child with autism (and in alternative embodiments, where also that child also has a de novo mutation coming from the father) then having a second child with autism by assessing the genetic content of the father's sperm, e.g., using a sperm donation from the father. In alternative embodiments, the genetic content of the sperm is determined using Digital Droplet PCR (ddPCR) or equivalents.

In alternative embodiments, methods provided herein address needs arising from the major push towards clinical sequencing inside and outside of the United States, and provides a method for genetic diagnosis that can become standard for many conditions. In alternative embodiments, methods provided herein provide an appropriate risk assessment to the affected families, and thus addressing an important concern, e.g., by assessment of de novo mutations in the paternal sperm.

In alternative embodiments, methods provided herein can assess de novo genetic variations, which are thought to be one of the major contributors to congenital human disease across a variety of conditions that include, but are not limited to, congenital heart disease, intellectual disability, autism spectrum disorders, and schizophrenia (see, e.g., Fromer et al., 2014; Homsy et al., 2015; Huguet et al., 2013; Vissers et al., 2010). In alternative embodiments, methods provided herein can assess de novo genetic variations that contribute to early and late miscarriages which impose an emotional and physical burden on pregnant couples (see, e.g., Carss et al., 2014).

Currently de novo variations are widely thought to occur at the final stages of sperm cell division, resulting in two main assumptions: first, these are individual events that are independent and do not influence risk for subsequent inheritance; second, they are by definition unpredictable, i.e. not amenable to genetic testing. However, our data show that these assumptions appear to be incorrect. A significant portion of de novo variants that we have assayed are detectable in sperm, but not in blood or saliva derived from the father, at percentages that far exceed what would be seen if the variants arise during the final stages of sperm division. Furthermore, the percentages of sperm cells carrying these de novo variants are high enough to confer significant disease risk, and in alternative embodiments methods provided herein can assess this risk.

These results have several important implications: 1) sperm, as the agent that transmits the genetic information to a child, should be the primary sample analyzed for genetic testing—as with alternative embodiments provided herein; 2) recurrence risks of de novo mutations may have to be assessed differently in clinical practice (i.e. negative results using parental blood ought to be supplemented with testing of sperm cells to provide a more accurate risk assessment—as with alternative embodiments provided herein, where testing of sperm cells is supplemental to the testing of parental blood or other non-sperm sample); and 3) genetic testing as provided herein has the power to predict a subset of de novo cases, which could have tremendous implications for health care and disease prevention.

In alternative embodiments, methods provided herein comprise use of a ‘haploinsufficiency-ome’ or other disease specific ‘omes’ for gene sequencing of de novo disease mutations. Other gene panels used in methods provided herein include intellectual disability or autism genes, and there is only partial overlap of genes on these panels with genes in the ‘haploinsufficiency-ome’ provided herein. Further, genes in ‘haploinsufficiency-ome's as provided herein were selected only in part due to their implication in these diseases. In alternative embodiments, methods provided herein apply use of specific gene lists based upon their likelihood to cause disease when haploinsufficient.

In alternative embodiments, methods provided herein comprise use of gene panels developed for sperm sequencing, noting that other sequencing efforts used by fertility experts utilize only samples from parents' blood, or from the fertilized embryo. In alternative embodiments, methods provided herein comprise use of sperm genetic assessment for the prevention of diseases and conditions, including pediatric disease.

In alternative embodiments, methods provided herein comprise use of gonadal mosaicism from sperm as a diagnostic tool. Current applications of tests of mosaicism are almost exclusively limited to the field of cancer. Provided herein are clinical applications of tests of mosaicism outside of the cancer field.

In alternative embodiments, methods provided herein comprise use of ddPCR for genetic counseling in the realm of congenital disease.

In alternative embodiments, methods provided herein provide a prenatal diagnosis that is performed at a time prior to conception using DNA from germ cells, wherein current applications of prenatal testing involve assessment of parental blood samples, or sampling the fertilized embryo prior to implantation in the practice of IVF. In alternative embodiments, methods provided herein can replace or supplement prenatal genetic diagnosis (PGD), which involves assessment of single genes mutations from single cells extracted from a fertilized embryo. In alternative embodiments, methods provided herein can replace or supplement prenatal genetic screening (PGS), which involves the assessment of chromosomal counts from single cells as well. In alternative embodiments, methods provided herein assess parental germ cell for genetic lesions that could be different from blood.

In alternative embodiments, methods provided herein provide a risk assessment for disease in children that is determined from sperm, where current assessments for risk are based upon paternal age and morphology of sperm. If there is advanced paternal age or if the sperm generally show abnormal morphology, then the current practice is to perform IVF and then implant only female embryos (because there is a lower risk of autism in female offspring), or utilize a sperm donor. In alternative embodiments, methods provided herein can replace or supplement can determine which males are at higher vs. lower risk, which can help prospective parents to make more informed decisions.

In alternative embodiments, methods provided herein can take into account paternal age when determining risk of disease in offspring. The current assumption in the relevant literature is that the vast majority of de novo variants are due to age-dependent defects in paternal sperm, but the current practice does not allow assessment of de novo mutations in genetic counseling. In alternative embodiments, methods provided herein is a sequencing method of the germ cells in order to determine which older males are at high risk for children with disease.

We utilized an existing cohort of children with autism on whom we identified a de novo mutation coming from the father in order to assess if the sperm from the father carried the same mutation in detectable levels. This method can be used to assess risk of the couple having a second child with autism after a first child is diagnosed, by assessing sperm donation from a father, using the very sensitive method of ddPCR.

In alternative embodiments, the genetic content of the sperm is determined using Digital Droplet PCR (ddPCR), which is a digital PCR variation where the PCR solution is divided into smaller reactions through a water oil emulsion technique, which are then made to run PCR individually. Digital polymerase chain reaction (digital PCR, DigitalPCR, dPCR, or dePCR), is a polymerase chain reaction variation that can be used to directly quantify and clonally amplify nucleic acids strands including DNA, cDNA or RNA.

In alternative embodiments, this method of sperm sampling is used to screen for mutations in the list of 1000+ putative autism genes (what we call the ‘autism-ome’) to determine the risk of autism. This could be applied to couples that have already had a child with autism but that do not yet know the mutation, or it could be applied to couples without a previous child with autism, but where a couple wants to assess their personalized risk.

In alternative embodiments, this method of sperm sequencing could be used to screen any list of genes including the whole ‘haploinsufficiency-ome’ to assess the risk of de novo mutations being transmitted. This method could be used to sequence all or part of the ‘exome’ at read depth of 1000 fold or greater, at a reasonable cost and high predictive power.

In alternative embodiments, PCR primer pairs and a ddPCR method were used to detect mutations in sperm DNA samples of fathers of autistic children, where it was known what mutation caused the autism in the child. First, or de novo, mutations were identified in sperm samples from a group of fathers of some of the autistic children. Some sperm samples carried the same mutation that caused the autism in the child of the father; however, a blood sample from the same father was negative for this same mutation—thus identifying the mutation as a de novo mutation. We found that around 10% to 30% of sperm cells from the father carried the mutation. Thus, these couples are then at high risk for recurrence of autism in a potential (or existing) sibling of the autistic child. We also identified some sperm samples from other fathers (of autistic children) that were not carrying any evidence of the mutation that caused autism in their child. These couples are then at low risk for recurrence of autism in a second (an additional) child.

In alternative embodiments, methods provided herein allows prediction of the risk of autism or other de novo mutation diseases arising from male sperm. By assessing the sperm of males planning to have children using high read depth sequencing, e.g., using ddPCR, the risk that a fetus will receive a mutation that is present in the father's sperm but not present in the rest of his body can be assessed. Thus, exemplary methods as provided herein are a direct way of sampling sperm to detect the personalized risk of having a child with a hereditable disease.

In alternative embodiments, these methods work with specific mutations (i.e., alleles), or with a whole panel of genes contributing to or associated with a specific disease, such as autism (e.g., the ‘autism-ome’), or with a whole panel of genes that produce haploinsufficient birth defects or other diseases when one copy of the gene is missing (i.e., the ‘haploinsufficiency-ome’).

In alternative embodiments, methods provided herein provide an individualized risk assessment that can help couples decide whether to conceive naturally or through artificial insemination, through preimplantation genetic diagnosis, or through adoption. In alternative embodiments, methods provided herein are able to reduce the risk of autism or other haploinsufficient diseases like schizophrenia, congenital heart disease, genetic syndromes, etc, in the general population, for example, reduce the risk by perhaps as much as half.

In alternative embodiments, compositions, e.g., kits, and methods provided herein comprise technology to ship and receive sperm samples from males through the postal service, to isolate DNA from sperm, to perform DNA sequencing at specific alleles or using specific gene panels, to annotate these genetic changes, and to produce a report that has high positive and negative predictive value. In alternative embodiments, methods provided herein utilize well-accepted methods from the cancer and human genetics field including ddPCR, panel deep sequencing, and risk assessment. ddPCR methodology can generate sequence of a particular allele on 10,000 individual cells in a single PCR reaction, to allow for high sensitivity and specificity of the mutation from a mixture of cells.

It is well established that the average age of couples conceiving children is advancing in society, and with this comes an elevated risk of de novo mutations from the father. One study reported that men aged 50 years and older are twice as likely as men under age 30 to have a child with autism. At the same time, sequencing studies suggest that each year a man ages, he passes an estimated two more de novo, or spontaneous mutations, to children he sires. Therefore, there is a great health care need to offer personalized risk assessments to couples planning pregnancies.

Furthermore, whole exome and whole genome sequencing is becoming part of the routine assessment of children with birth defects or neurocognitive defects like autism or intellectual disability or epilepsy. Therefore, the number of genetically diagnosed cases is likely to continue to rise dramatically in the next 10 years, to a point where all or nearly all cases will receive a genetic diagnosis. This will result in many families seeking and needing information about genetic risk in future children.

In alternative embodiments, methods as provided herein is used in cases where the mutation is known from a first affected child; the risk of having another child with the same mutation can be precisely defined with methods as provided herein. In alternative embodiments, methods as provided herein can perform risk assessment from sperm in cases where the mutation is already known from a first affected child.

In alternative embodiments, methods as provided herein are used in cases without a prior family history or even with a positive family history, but where the mutation is not known; however, the risk of future pregnancies can be much more precisely defined by genetic profiling of the father's sperm. This could be applied to any range of disorders where de novo mutations are the cause, and these include: trinucleotide expansions—in order to assess stability of repeats; neurocutaneous disease to exclude de novo mutations; congenital heart disease risk assessment; schizophrenia risk assessment; intellectual disability assessment; and, chromosomal rearrangement risk assessment, noting that the list will continue to grow as new de novo genetic mutations are identified.

In alternative embodiments, methods as provided herein incorporate a broad screening of sperm to include various ‘omes’ such as ‘autism-ome’, ‘Congenital heart disease-ome’, ‘Schizophrenia-ome’, ‘Intellectual disability-ome, etc. With such a profile assessing risk of de novo mutation being transmitted, if the risk is deemed to be low, couples gain increased peace-of-mind prior to conception. If the risk is deemed to be high, the family can opt instead to use a donor or pre-implantation genetic diagnosis.

### Methods for Inferring Disease Risk in Offspring by Detection of Somatic Mosaic Variants in Parental Sperm or Somatic Tissues

In alternative embodiments, provided are compositions, including products of manufacture and kits, and methods, for determining the risk of inheritance of a genetic defect or trait in a younger child or a potential sibling, wherein the younger child or potential sibling to be assessed for inheritance of the genetic defect or trait has a sibling already diagnosed with that genetic defect or trait. In alternative embodiments, the disease caused by the genetic defect or trait is autism, schizophrenia, heart disease, congenital heart disease or a neurocutaneous disease.

In alternative embodiments, provided are methods for the detection of single nucleotide variants (SNVs) and structural variants (SVs, including deletions, insertions and inversions) and the application of these methods for predicting in men or women their risk for having a child with a genetic disorder. In alternative embodiments, provided are applications these methods as a non-invasive pre-natal testing (NIPT) to determine whether a fetus is carrying a genetic variation, e.g., a high-risk mutation that may cause a genetic disorder or undesired trait.

In alternative embodiments, these methods comprise assessing DNA samples from sperm or blood from parents planning to have a child using polymerase chain reaction-(PCR-) based or whole genome sequencing (WGS) methods for the risk that the new child (a fetus) will receive a mutation that is present as a somatic mutation in one or both of the parents. In alternative embodiments, these methods produce an individualized risk assessment that can help couples decide whether to conceive naturally or through artificial insemination, or through preimplantation genetic diagnosis; or, alternatively, have another child by adoption.

In alternative embodiments, these methods are able to reduce the risk of autism or other haploinsufficient diseases like schizophrenia, congenital heart disease, genetic syndromes, etc, in the general population.

In alternative embodiments, these methods also comprise shipping and receiving sperm samples from males through public delivery (e.g., the postal service), and using these samples to isolate DNA, to perform DNA sequencing at specific alleles or at specific gene panels, to annotate these genetic changes, and to produce a report that has high positive and negative predictive value.

In alternative embodiments, these methods are applicable when a prospective father (male) parent has been identified as the genetic carrier of a DNA variation, e.g., a high-risk mutation.

In alternative embodiments, provided are methods for making a genetic assessment of sperm, i.e., for using sperm as a way to assess risk of an inherited, or genetically transmitted disease or a trait, e.g., a childhood disease or a trait. In alternative embodiments, provided are methods that help couples that have had a child with a genetic disease or trait due to a de novo genetic mutation know with a high degree of certainty that a next child will also have that same genetic disease or trait. In alternative embodiments, these methods can be used as a non-invasive prenatal test (NIPT) for detecting a small number of extra chromosomes that can form viable offspring (e.g., as with Trisomy 21, 18, 13), or to detect single nucleotide variants (SNVs) or structural variants (SVs).

In alternative embodiments, methods provide positive and negative predictive values for every mutation detected and profiled; for example, a numerical assessment of risk can be provided as a personalized report that will be useful for health care professionals (genetic counselors, reproductive endocrinologists, fertility specialists, pediatricians and geneticists) and couples.

In alternative embodiments, provided are methods that provide a genetic assessment of risk of recurrence of a DNA mutation, e.g., a genetic variation or defect, in a second child when an earlier child of the same parents has the same DNA mutation, e.g., a genetic variation or defect. Because males produce 1500 sperm cells per second throughout life, and most of these individual cells are thought to derive from a collection of perhaps a few thousand sperm stem cells, by assessing a collection of thousands of sperm the sensitivity of assays as provided herein to assess for these mutations is very high. The sensitivity is not 100% though, and there is still the possibility that a single sperm carries a genetic mutation that can cause disease. The revolution in genetics has led to insights that form the basis of our invention. There have been numerous reports attributing de novo mutations as a cause for birth defects such as congenital heart disease, neurocutaneous disorders, autism and schizophrenia [1-5]. Risk of recurrence in families is in the range of 10%. For instance, germline mosaicism was detected in 11.6% of parents of children with Duchenne/Becker muscular dystrophy [6]. Recent studies have begun to address somatic rather than germline mosaicism. For instance, in one study of 100 families with de novo mutation, there was evidence of somatic mosaicism in one of the parents in 4 cases assessed from blood (25087610). The difference from our approach is that somatic mosaicism rates are very rare for transmitted mutations, and thus not useful to determine personal risk at scale. There are also studies that perform exome sequencing on the fetus when found to have a structural defect based upon ultrasound, where between 10-27% of cases had likely genetic diagnosis made prior to birth [7]. The difference from our approach is that families want this information prior to conceiving a fetus with a genetic mutation. There have been reports in the literature of an increased risk of psychiatric disorders such as autism and schizophrenia with increased age of the father at the time of conception [8-14]. There are papers that propose mechanisms my which de novo mutations in sperm lead to over-proliferation of specific clones, which has been proven in only a few examples [15], which could be one mechanism by which age influences the rate of de novo sperm mutations. Most but not all risk is thought to result from an age-dependent effect on the accumulation of de novo mutations, whereas some of the increased risk of autism in older fathers due to de novo mutations was postulated to be from age-independent effects, which remains an active area of research [16].

In alternative embodiments, method can be used to assess risk of a couple who has a child with autism (and in alternative embodiments, where also that child also has a de novo mutation coming from the father) then having a second child with autism by assessing the genetic content of the father's sperm, e.g., using a sperm donation from the father. In alternative embodiments, the genetic content of the sperm is determined using Digital Droplet PCR (ddPCR) or equivalents.

In alternative embodiments, methods provided herein address needs arising from the major push towards clinical sequencing inside and outside of the United States, and provides a method for genetic diagnosis that can become standard for many conditions. In alternative embodiments, methods provided herein provide an appropriate risk assessment to the affected families, and thus addressing an important concern, e.g., by assessing the risk that a second, or subsequent, child inherits a trait, disease or condition already inherited by an earlier sibling.

In alternative embodiments, methods provided herein can assess de novo genetic variations, which are thought to be one of the major contributors to congenital human disease across a variety of conditions that include, but are not limited to, congenital heart disease, intellectual disability, autism spectrum disorders, and schizophrenia (see, e.g., Fromer et al., 2014; Homsy et al., 2015; Huguet et al., 2013; Vissers et al., 2010). In alternative embodiments, methods provided herein can assess de novo genetic variations that contribute to early and late miscarriages which impose an emotional and physical burden on pregnant couples (see, e.g., Carss et al., 2014).

In alternative embodiments, methods provided herein can replace or supplement prenatal genetic diagnosis (PGD), which involves assessment of single genes mutations from single cells extracted from a fertilized embryo. In alternative embodiments, methods provided herein can replace or supplement prenatal genetic screening (PGS), which involves the assessment of chromosomal counts from single cells as well. In alternative embodiments, methods provided herein assess parental germ cell for genetic lesions that could be different from blood.

In alternative embodiments, these methods are employed as a means for non-invasive prenatal testing (NIPT), in which a specific mutation of interest can be detected by PCR or genome sequencing of circulating DNA in the blood of a pregnant mother. Detection of a high-risk mutation would provide the mother with the option to make reproductive decisions based on genetic information or to make preparations for the immediate care of a child born with a genetic disorder.

In alternative embodiments, methods provided herein can take into account paternal age when determining risk of disease in offspring. The current assumption in the relevant literature is that the vast majority of de novo variants are due to age-dependent defects in paternal sperm, but the current practice does not allow assessment of de novo mutations in genetic counseling.

In alternative embodiments, compositions, e.g., kits, and methods provided herein comprise technology to ship and receive sperm samples from males through the postal service, to isolate DNA from sperm, to perform DNA sequencing at specific alleles or using specific gene panels, to annotate these genetic changes, and to produce a report that has high positive and negative predictive value. In alternative embodiments, methods provided herein utilize well-accepted methods from the cancer and human genetics field including ddPCR, panel deep sequencing, and risk assessment. ddPCR methodology can generate sequence of a particular allele on 10,000 individual cells in a single PCR reaction, to allow for high sensitivity and specificity of the mutation from a mixture of cells.

It is well established that the average age of couples conceiving children is advancing in society, and with this comes an elevated risk of de novo mutations from the father. One study reported that men aged 50 years and older are twice as likely as men under age 30 to have a child with autism. At the same time, sequencing studies suggest that each year a man ages, he passes an estimated two more de novo, or spontaneous mutations, to children he sires. Therefore, there is a great health care need to offer personalized risk assessments to couples planning pregnancies.

Furthermore, whole exome and whole genome sequencing is becoming part of the routine assessment of children with birth defects or neurocognitive defects like autism or intellectual disability or epilepsy. Therefore, the number of genetically diagnosed cases is likely to continue to rise dramatically in the next 10 years, to a point where all or nearly all cases will receive a genetic diagnosis. This will result in many families seeking and needing information about genetic risk in future children.

In alternative embodiments, methods as provided herein is used in cases where the mutation is known from a first affected child; the risk of having another child with the same mutation can be precisely defined with methods as provided herein. In alternative embodiments, methods as provided herein can perform risk assessment from sperm in cases where the mutation is already known from a first affected child.

In alternative embodiments, methods as provided herein are used in cases without a prior family history or even with a positive family history, but where the mutation is not known; however, the risk of future pregnancies can be much more precisely defined by genetic profiling of the father's sperm. This could be applied to any range of disorders where de novo mutations are the cause, and these include: trinucleotide expansions—in order to assess stability of repeats; neurocutaneous disease to exclude de novo mutations; congenital heart disease risk assessment; schizophrenia risk assessment; intellectual disability assessment; and, chromosomal rearrangement risk assessment, noting that the list will continue to grow as new de novo genetic mutations are identified.

All Droplet Digital PCR (ddPCR) technology and protocols and all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

Droplet Digital PCR (ddPCR™) Systems used to practice methods, kits and products of manufacture as provided herein can be or comprise e.g., Droplet Digital PCR (ddPCR™) Systems, including QX200™ or AutoDG™ Droplet Digital PCR Systems (Bio-Rad Hercules, Calif.).

In alternative embodiments, once a male is identified as a potential client, a sterile collection tube (e.g., Nalgene's 2 oz straight-sided polypropylene jar (Cat #341416)) is send to the client. The client produces a semen sample into the tube and ships it to the lab in a self-addressed stamped envelope, where it is received within 24 hours. New packages are checked into the lab and assessed for semen volume, color and potential contamination.

### DNA Extraction From Sperm

In alternative embodiments, extraction of sperm cell DNA from fresh ejaculate is performed as previously described (see e,g,, Wu et al., 2015). Any excess material is frozen (−80° C.) employing a TYB-based freezing medium (Irvine Scientific, 90128) according to the manufacturer's protocol. This frozen semen can be thawed and used instead of fresh ejaculate. Due to the dilution with freezing medium, however, yields will be at least 50% lower relative to the extraction of fresh ejaculate using the same starting volume.

In alternative embodiments, sperm cells are isolated by centrifugation over an isotonic solution (90%) (Sage/Origio, ART-2100; Sage/Origio, ART-1006) using up to 2 mL of the sample. Following a washing step, quantity and quality are assessed using a cell counting chamber (Sigma-Aldrich, BR717805-1EA). Cells are then pelleted and lysis is performed by addition of RLT lysis buffer (Qiagen, 79216), Bond-Breaker TCEP™ solution (Pierce, 77720), and 0.2 mm stainless steel beads (Next Advance, SSB02) on a Disruptor Genie™ (Scientific Industries, SI-238I). Lysate is then processed using reagents and columns from an AllPrep™ DNA/RNA Mini Kit (Qiagen, 80204). Concentration of the final eluate is assessed employing standard methods. Typical concentrations range from 10-300 ng/μl (note that even lower concentrations have been successfully used for ddPCR analysis). Sperm extracted DNA is subsequently stored on −20° C. until use.

### Nested PCR Assay to Detect Somatic Mosaic Structural Variants

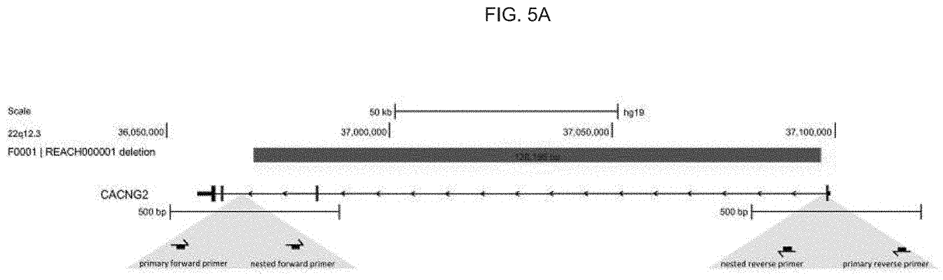

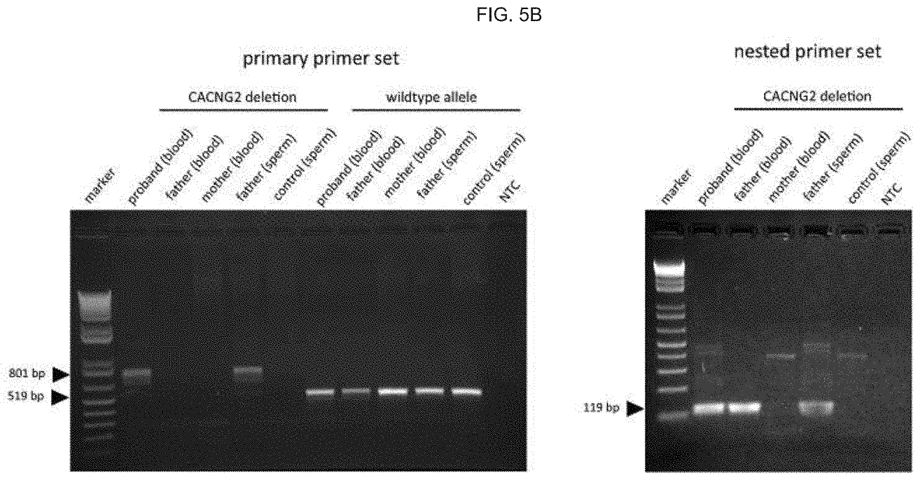

In alternative embodiments, the presence of a somatic mosaic allele is detected in DNA derived from sperm or somatic tissues by using series of multiple PCR reactions using a primary set of primers that are specific to the mutant allele and a secondary set of nested primers that target the amplicon that is produced from the mutant allele (FIG. 5A). PCR is performed according to standard methods. For instance, We have reduced this method to practice by demonstrating the detection of a germline deletion of CACNG2 in the proband of family F0001 from the REACH study {Brandler 2015} and detecting the same deletion as a somatic mosaic variant sperm and blood from the child's father (FIG. 5B). In the example provided, the deletion is present at sufficient frequency in the sperm sample to enable it's detection using the primary primer set; however, the frequency of the deletion in the blood was low, and it could only be detected by performing a second PCR amplification using the nested primers.

FIG. 5A-B schematically and graphically illustrates detection of a somatic mosaic Structural Variant in paternal sperm and blood by nested PCR. FIG. 5A schematically illustrates: a de novo deletion of the gene CACNG2 as originally detected by 30× whole genome sequencing blood-derived DNA from the proband (REACH0001) in family F0001 from our ongoing genetic studies of autism. The mutation was found to be absent from the genomes of the mother and father. Two sets of primers were designed to specifically amplify the deletion breakpoint, a primary set and a “nested” set that is contained within the primary amplicon. FIG. 5B schematically illustrates data from a PCR assay using the primary set of primers that detects the CACNG2 deletion in blood derived DNA from the proband and sperm-derived DNA from the father. A sequential amplification of the primary products using nested primers detects the deletion in the sperm and blood of the father, confirming a relatively high frequency of the mosaic variant in paternal sperm and a relatively low frequency of the variant in patneral blood.

Digital Droplet PCR (ddPCR) Assay

In alternative embodiments, the mutant sequence (SNPs, indels or SV breakpoints) are detected as a somatic mosaic variant in DNA derived from sperm, or somatic tissues using a ddPCR assay.

Using the Primer3Plus™ web interface (Koressaar and Remm, 2007; Untergasser et al., 2012; Untergasser et al., 2007), primers targeting a short DNA fragment (an amplicon of 100 bp and shorter if possible) and probes (20 bp or shorter if possible) for the mutant sequence are designed. Optionally, a 2^{nd} amplicon and probe for the wild type (reference) sequence is designed. Probes are designed to target the mutation site or SV breakpoint junction. Probes for the mutant and wild-type alleles are also adjusted so that melting temperatures (Tm) are matched. Following the successful identification of primer and probe sets, specificity of the primers is assessed using Primer-BLAST (Ye et al., 2012).

Custom primer and probe mixes (e.g., primer to probe ratio of 3.6) can be used. By convention, mutant probes are ordered using the FAM dye, whereas wild-type probes are labeled with HEX. In parallel, as a positive control, a gBlock gene fragment (IDT) of the interrogated locus with the mutation of interest is designed. Within the designed amplicon three bases that lie outside the probe sequences are scrambled to act as a potential targeting point to identify gBlock contamination if necessary. Alternatively, a distinct sequence with an already established ddPCR strategy can be included outside the amplicon.

In alternative embodiments, once received, primer/probe mixes are resuspended according to the manufacturer's protocol to yield a 20× concentrate. gBlock fragments are resuspended in nuclease-free ddH₂O and subsequently diluted to match the gene copies present in the control reaction.

ddPCR is performed on a BioRad platform, using a QX200™ droplet generator, a C1000™ touch cycler, a PX1™ PCR Plate Sealer, and a QX200™ droplet reader.

ddPCR reactions are set up in the following way: 8 μl of DNA solution (30-50 ng of genomic DNA total), 1 μl of the mutant primer/probe mix, 1 μl of the wild type primer/probe mix, 10 μl ddPCR Supermix™ (BioRad, 1863024). Following mixing, the ddPCR reaction and droplet generation oil (BioRad, 1863005) are transferred to a cartridge (BioRad, 1864008) to generate reaction droplets according to the manufacturer's instructions. The emulsified solution is transferred onto a PCR plate (Eppendorf, 951020346) and the PCR protocol is run on the thermocycler (Appendix 2, see below). Following PCR, reactions are analyzed on the droplet reader using QuantaSoft™ (BioRad).

For each reaction, at least three independent runs are performed: First, a gradient PCR determines the optimal annealing temperature for each assay using control DNA supplemented with gBlock (i.e. a PCR template based upon a synthesized oligonucleotide that contains the mutation being assessed) at a 1:10 ratio (number of copies) and non-template controls (NTC). The optimal temperature is defined as the one with the most orthogonal separation along the two axes while allowing maximal distinction of baseline versus signal. Second, a test run employing the chosen temperature is performed on positive controls with the gBlock at 1:1 and 1:10 ratios, control DNA and NTC. This helps to determine the cutoffs and the false positive rates for the mutation. Third, the experimental run is performed, in which the extracted sperm sample (as a technical triplicate), DNA extracted from control sperm, and NTC will be analyzed. This experiment can be extended by including paternal and maternal samples derived from blood or saliva and patient DNA. Alternatively, these analyses are performed only in case of detected mosaicism in a later run.

The QuantaSoft™ and QuantaSoft Analysis Pro™ software packages (BioRad) allow direct quantification of abundance of mutant versus wild-type allele. The sensitivity of a given assay is variable, however, mosaicism of above 0.1% could be confidently called across all tested conditions. For positive samples, an independent biological replicate is necessary to confirm mosaicism. Risk of transmitting the variant of interest to offspring equals the fractional abundance of the variant in sperm (mutant/[mutant+wild type]).

### Exemplary PCR Protocol

1. 95° C. for 10 minutes
2. 94° C. for 30 seconds
3. X° C. for 1 minute (Temperature depends on assay)

Repeat 2 and 3 for a total of 40 cycles
4. 98° C. for 10 minutes
Steps 2-4 are done with a temperature ramp of 2.0° C./second.

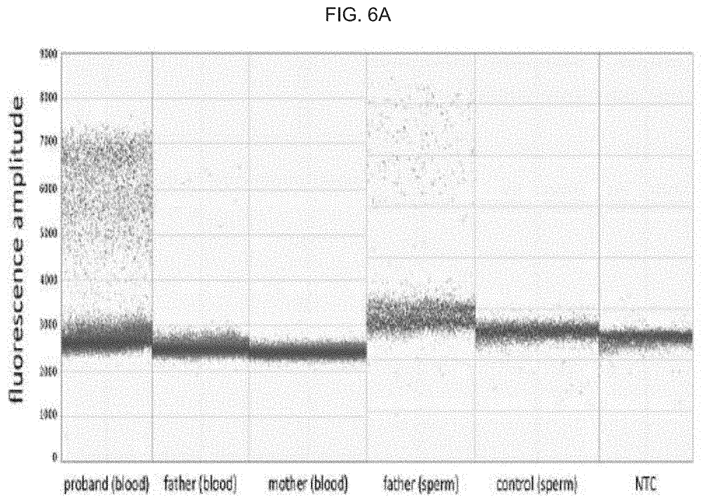

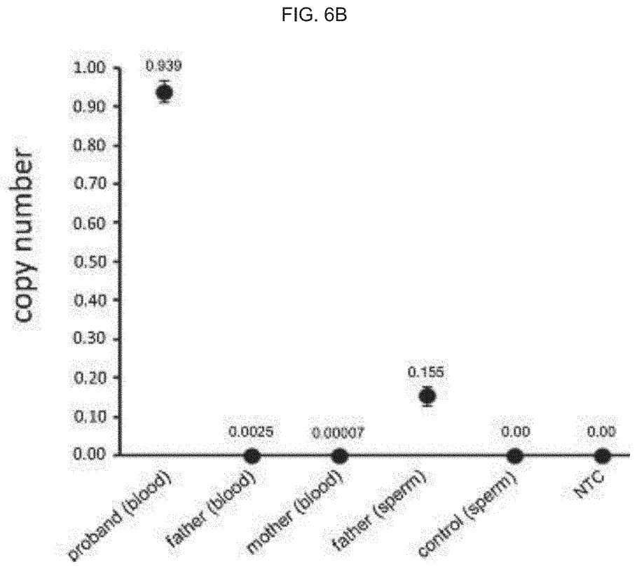

We have reduced this method to practice by quantifying the number of copies of the CACNG2 deletion allele that are present in paternal sperm and blood in REACH family F0001 (FIG. 6). Estimates indicate that the deletion is present in one copy per diploid genome in blood-derived DNA from the proband, and it is present in 0.155 and 0.0023 copies per diploid genome in the father's sperm and blood respectively. Thus, we estimate that the deletion is present in approximately 7.8% of sperm, indicating that the probability of transmitting this deletion to subsequent offspring is approximately 7.8%. In addition, ddPCR confirmed a low (approximately 1.2%) frequency of the mutation in the father's blood, consistent with the previous results using the nested PCR assay.

### Detection of Somatic Mosaic Variants by Whole Genome Sequencing

In alternative embodiments, a somatic mosaic variant (SNP, indel or SV breakpoint) is detected in DNA derived from sperm or somatic tissues by deep whole genome sequencing. Sequencing library preparation is performed using (1) Illumina's standard library preparation protocols or (2) a large-insert library constructed using currently methods such “jumping libraries” (PMID 21473983) or fosmid libraries (PMID: 22800726). Sequencing is then performed using Illumina™ short-read sequencing technology (150 bp paired ends at mean coverage of between 50 and 1000×).

Detection and quantification of the somatic mosaic variant is then achieved by determining the relative proportion of reads or inserts that support the mutant allele relative to the total number of reads that support the wild-type allele. “Supporting” reads are defined as individual or paired-end reads with allele-specific signatures; for example that align to multiple breakpoints of a SV or that contain the mutant allele (SNP or indel). By quantifying the proportion of reads that support the mutant sequence, the relative proportion of chromosomes that carry the mutant allele. We have reduced this approach to practice by performing whole genome sequencing of multiple family members of REACH family F0001 and quantifying the relative proportion of chromosomes that carry the deletion of CACNG2. Whole genome sequencing was performed on blood-derived DNA from the proband, mother and father at a depth of 100-200×, and sperm derived DNA from the father was sequenced at a depth of 200×. In the proband's genome, 41% of reads supported the mutant allele. This is similar to the average alternative-allele frequency genome wide for heterozygous variants, and is consistent with the CACNG2 deleting being present in all cells (expected frequency close to 50%). In the father's sperm and blood, the CACNG2 deletion allele was supported by 3.6% and 0.5% of reads respectively, consistent with the frequency estimates from ddPCR. The deletion was not detected in the mother's genome. Recalibrating these estimates to the average alternative-allele frequency genome wide, gives an estimate of a 5% deletion frequency in the father's sperm.

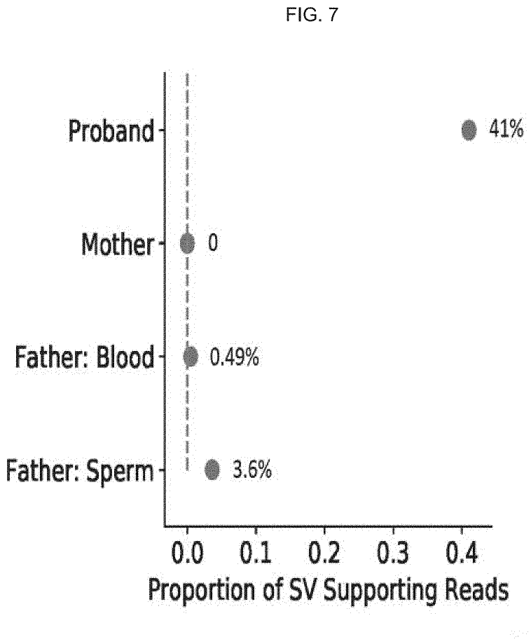

FIG. 7 graphically illustrates data from the detection and quantification of a somatic mosaic Structural Variant by whole genome sequencing. Split reads and discordant paired-end reads are quantified from whole genome sequence alignments and the proportion of reads supporting the mutant allele are determined. Implementation of this approach to blood-derived DNA from REACH family F0001 and sperm-derived DNA from the father, provides an estimate of the proportion of cells that contain a deletion of the gene CACNG2.

We have applied this method to larger whole genome sequencing (WGS) dataset of blood-derived genomic DNA samples from 133 families and detected an additional 8 somatic mosaic SVs. From these data we estimate that approximately 6% ( 8/133; CI=2.8-11.4%) of SVs that are detected as de novo mutations in offspring, also display either high-level somatic mosaicism in the offspring or low-level somatic mosaicism in a parent.

### Detection of a Somatic Mosaic Variant by Restriction Site Mutation (RSM) Assay

In alternative embodiments, a somatic mosaic variant can be detected in sperm or somatic tissues using a restriction enzyme that targets a recognition sequence that overlaps with the mutation site (PMID: 10473646). If the mutant sequence eliminates the restriction site, the mutation can be detected by (1) eliminating the wild-type allele by complete digestion of the genomic DNA followed by amplification of the mutant sequence by PCR using primers that flank the mutation site. The sensitivity of the restriction site mutation assay can be further enhanced by performing multiple cycles of digestion and PCR using multiple sets of nested primers. Some restriction enzymes are blocked by CpG methylation, and this confounding issue can be addressed by incorporating initial PCR steps prior to the first digestion. An alternative approach to RSM that does not require the mutation to overlap a specific recognition sequence is to (1) stimulate the wild-type allele to form heteroduplex DNA by competitive hybridization with an oligonucleotide containing the mutant allele, followed by (2) digestion with T4 endonuclease VII.

### A Non-Invasive Prenatal Test (NIPT) for the Presence of a Mutant Allele in Fetal DNA

In alternative embodiments, all of the above techniques can be applied to cell-free circulating fetal DNA derived from peripheral blood of the pregnant mother. This assay would be applicable to pregnancies that are determined to be high-risk based on the presence of a somatic mosaic mutation in the father's sperm or based on developmental abnormalities observed by fetal ultrasound.

A 5 sample of peripheral blood is obtained from the pregnant mother, and preparation of cell free DNA from maternal blood is performed as follows. Plasma is obtained from peripheral blood by centrifugation at 16,000×g for 10 minutes. Cell-free DNA is then purified from <1 ml of plasma using commercially-available preparation kit, such as the GenMag circulating DNA from Plasma Kit (http://genmagbio.com).

### Mutations in Fetal DNA

Detection of a disease-causing mutation in fetal DNA is then carried out by testing cell-free DNA using one of the methods described above, including ddPCR, nested PCR, RSM, or whole genome sequencing assays.

### REFERENCES

1. Dies, K. A. and M. Sahin, Genetics of neurocutaneous disorders: basic principles of inheritance as they apply to neurocutaneous syndromes. Handb Clin Neurol, 2015. 132: p. 3-8.

2. Fromer, M., et al., De novo mutations in schizophrenia implicate synaptic networks. Nature, 2014. 506(7487): p. 179-84.

3. Zaidi, S., et al., De novo mutations in histone-modifying genes in congenital heart disease. Nature, 2013. 498(7453): p. 220-3.

4. Neale, B. M., et al., Patterns and rates of exonic de novo mutations in autism spectrum disorders. Nature, 2012. 485(7397): p. 242-5.

5. Sanders, S. J., et al., De novo mutations revealed by whole-exome sequencing are strongly associated with autism. Nature, 2012. 485(7397): p. 237-41.

6. Bermudez-Lopez, C., et al., Germinal mosaicism in a sample of families with Duchenne/Becker muscular dystrophy with partial deletions in the DMD gene. Genet Test Mol Biomarkers, 2014. 18(2): p. 93-7.

7. Carss, K. J., et al., Exome sequencing improves genetic diagnosis of structural fetal abnormalities revealed by ultrasound. Hum Mol Genet, 2014. 23(12): p. 3269-77.

8. McGrath, J. J., et al., A comprehensive assessment of parental age and psychiatric disorders. JAMA Psychiatry, 2014.71(3): p. 301-9.

9. Pedersen, C. B., et al., The importance of father's age to schizophrenia risk. Mol Psychiatry, 2014. 19(5): p. 530-1.

10. Frans, E. M., et al., Autism risk across generations: a population-based study of advancing grandpaternal and paternal age. JAMA Psychiatry, 2013. 70(5): p. 516-21.

11. Kong, A., et al., Rate of de novo mutations and the importance of father's age to disease risk. Nature, 2012. 488(7412): p. 471-5.

12. Hultman, C. M., et al., Advancing paternal age and risk of autism: new evidence from a population based study and a meta-analysis of epidemiological studies. Mol Psychiatry, 2011. 16(12): p. 1203-12.

13. Petersen, et al., Paternal age at birth of first child and risk of schizophrenia. Am J Psychiatry, 2011. 168(1): p. 82-8.

14. Malaspina, D., et al., Advancing paternal age and the risk of schizophrenia. Arch Gen Psychiatry, 2001. 58(4): p. 361-7.

15. Goriely, A., et al., “Selfish spermatogonia) selection”: a novel mechanism for the association between advanced paternal age and neurodevelopmental disorders. Am J Psychiatry, 2013. 170(6): p. 599-608.

16. Gratten, J., et al., Risk of psychiatric illness from advanced paternal age is not predominantly from de novo mutations. Nat Genet, 2016. 48(7): p. 718-24.

17, Bianchi et al., Fetal gender and aneuploidy detection using fetal cells in maternal blood: analysis of NIFTY I data. National Institute of Child Health and Development Fetal Cell Isolation Study. Prenat Diagn. 2002 July; 22(7):609-15.

18. Guissart et al., J Cyst Fibros. Non-invasive prenatal diagnosis (NIPD) of cystic fibrosis: an optimized protocol using MEMO fluorescent PCR to detect the p.Phe508del mutation. 2017 March; 16(2):198-206. doi: 10.1016/j jcf.2016.12.011. Epub 2016 Dec. 28.

A number of embodiments of the invention have been described. Nevertheless, it can be understood that various modifications may be made without departing from the spirit and scope of the invention. Accordingly, other embodiments are within the scope of the following claims.

### EXAMPLES

Unless stated otherwise in the Examples, all Droplet Digital PCR (ddPCR) technology and protocols and all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

Droplet Digital PCR (ddPCR™) Systems used to practice methods, kits and products of manufacture as provided herein can be or comprise e.g., Droplet Digital PCR (ddPCR™) Systems, including QX200™ or AutoDG™ Droplet Digital PCR Systems (Bio-Rad Hercules, Calif.).

The invention will be further described with reference to the examples described herein; however, it is to be understood that the invention is not limited to such examples.

### Example 1

A couple has a positive family history of a disease like autism, and known genetic cause that traces to a de novo mutation from the father, and would like to know the risk of the next child inheriting the same genetic mutation. Male or health care provider interested in knowing individual risk of transmitting a de novo mutation decides to become a customer in order to have risk of a child with genetic disease assessed. Company ABC ships a sterile collection cup. The customer produces a semen sample at home into this cup, then ships it back to ABC using the provided envelope. ABC assesses the sample to ensure that there are high-quality sperm, then extracts DNA. Note that it is not possible to assess this from father's DNA since the mutation is only present in the sperm.

In this example, the information about the prior mutation would be passed to the company, then a set of PCR primers designed and tested to amplify this allele. The father's sperm sample is then assessed for this mutation using ddPCR. Based upon the percent of sperm cells carrying the mutation, the personalized risk can be calculated. For instance, if 25% of sperm cells carry the mutation, then there is a 25% risk of the next fetus inheriting this mutation. Since we know that the mutation caused the disease in the first child, we can assign a high positive predictive value of near 100%. This would be a family where health care providers might suggest an alternative to natural conception. But for instance if 0% of sperm cells carry the mutation of the 10,000 assessed, we can say that there is less than 1:1000 chance that a similar sperm ejaculate from the father would transmit the mutation to future pregnancies. Of course, we cannot exclude this possibility entirely, and we cannot exclude that a different mutation could have developed in the male's testes, but the risk should be no greater than the general population risk of 1:100 for autism.

Methods as provided herein also can apply to, be used to detect, any other de novo genetic mutation causing disease in a child. For instance, if there is a child born in the family with congenital heart disease, or a chromosomal structural defect, where the mutation is known and comes from the father, methods as provided herein can screen for the single mutation today from sperm sample from the family to provide an individual risk assessment.

### Example 2

A couple has a positive family history of a disease like autism but unknown genetic cause, and would like to know the risk of the next child inheriting the same disease. Samples are collected, but in this instance the sperm DNA sample is assessed for a panel of genes we call the ‘autism-ome’, which is a list of about 1000 genes in which haploinsufficient mutations lead have a high predictive value of leading to autism. The next-generation sequencing produces read depth of about 1000 across the exonic regions of these 1000 genes, and profiles all likely deleterious mutations of high effect.

If the sperm sample contains a severe mutation in one of these in 25% of sperm cells, then there is a 25% risk of the next fetus inheriting this mutation. In this instance we do not know the exact risk that this mutation will cause disease, but we will only report back mutations with an odds ratio of over 80%.

Importantly, the list of high-confidence genes in lists like the ‘autism-ome’ continues to grow, and in the next 5 years, will probably have a very complete list. The read depth of 1000 should be sufficient to identify all but the lowest rate of mosaicism in the father's sperm, and thus if there is no evidence of damaging mutations in these 1000 genes, we can report that the risk of autism is substantially below the baseline risk of 1:100. In fact, now it is possible to produce an adjusted risk based upon parental age, knowing that about 2 additional exonic mutations are added for every year of paternal age past 30 in males. Depending upon the odds ratio of these two different mutations, methods as provided herein can set a specific personalized risk of having a diseased child, which is independent of the paternal age. Thus, the predictive values of the test will increase in fathers as they age.

### Example 3

A couple has no positive family history but wishes to minimize the risk of de novo mutation for diseases like congenital heart disease, autism, schizophrenia, neurocutaneous disease or others due to haploinsufficiency. This sort of example is going to become especially important is fathers of advanced age, which is becoming a trend in our society. This will also become especially important as the list of the ‘haploinsufficiency-ome’ gets better defined, and the risk of disease (i.e. the odds ratio) of specific gene mutations can be assessed with more precision in the future. Sperm would be sequenced for the approximately 2000 genes in the ‘haploinsufficiency-ome’ at read depth of 1000, to produce a personalized risk assessment. For instance, if one or two different mutations of high predicted effect are found each in 10% of sperm, then the risk of transmitting of either would be 10% and the risk of transmitting both would be 1% (assuming that they are not in linkage disequilibrium). Depending upon the odds ratio of these two different mutations, methods as provided herein can set a specific personalized risk of having a diseased child, which is independent of the paternal age.

### Example 4: Exemplary Protocol for Collection of Semen

Once a male is identified as a potential client, a sterile collection tube is send to the client. The client produces a semen sample into the tube and ships it to the lab in a self-addressed stamped envelope, where it is received within 24 hours. New packages are checked into the lab and assessed for semen volume, color and potential contamination. There are several steps in the collection that were optimized:
We optimized the method of collection of semen sample that can be used for DNA isolation.
We found that a telephone call to a husband and wife together is key to convey the importance of testing sperm for mutation.
We found that most brands of shipping containers are not suitable for shipping of sperm. After trying different types, we have determined that Nalgene's 2 oz straight-sided polypropylene jar (Cat #341416) is the best option.
After connecting with a potential study participant/client, we put into the mail an envelope containing consent forms and the semen collection vial with a set of instructions for production of sample and return shipping. Samples are shipped at ambient temperature.
We have had shipment of samples from across the US and have found some variability in sample quality and resultant quality of DNA.
We found that overnight shipping to the laboratory is important for good sample quality.
Once samples are registered into the laboratory, we proceed to DNA extraction within 1 day.

### DNA Extraction From Sperm

Extraction of sperm cell DNA from fresh ejaculate is performed as previously described (see e,g Wu et al., 2015). Any excess material is frozen (−80° C.) employing a TYB-based freezing medium (Irvine Scientific, 90128) according to the manufacturer's protocol. This frozen semen can be thawed and used instead of fresh ejaculate. Due to the dilution with freezing medium, however, yields will be at least 50% lower relative to the extraction of fresh ejaculate using the same starting volume.

In short, sperm cells are isolated by centrifugation over an isotonic solution (90%) (Sage/Origio, ART-2100; Sage/Origio, ART-1006) using up to 2 mL of the sample. Following a washing step, quantity and quality are assessed using a cell counting chamber (Sigma-Aldrich, BR717805-1EA). Cells are then pelleted and lysis is performed by addition of RLT lysis buffer (Qiagen, 79216), Bond-Breaker TCEP™ solution (Pierce, 77720), and 0.2 mm stainless steel beads (Next Advance, SSB02) on a Disruptor Genie™ (Scientific Industries, SI-238I). Lysate is then processed using reagents and columns from an AllPrep™ DNA/RNA Mini Kit (Qiagen, 80204). Concentration of the final eluate is assessed employing standard methods. Typical concentrations range from 10-300 ng/μl (note that even lower concentrations have been successfully used for ddPCR analysis). Sperm extracted DNA is subsequently stored on −20° C. until use.

PCR Amplification of Regions of Interest and ddPCR Reagent Design

In order to assess the conservation of the region surrounding the mutation of interest, PCR and Sanger sequencing are performed according to standard methods. The resulting sequence is compared to reference and any observed SNPs are taken into account for the subsequent design of the ddPCR assay.

Using the Primer3Plus™ web interface (Koressaar and Remm, 2007; Untergasser et al., 2012; Untergasser et al., 2007), amplicon and probes for wild-type and mutant are designed using the settings in Appendix 1, see below. Probes are designed within 15 base pairs (bp) up- and 15 bp downstream of the mutation and adjusted, so melting temperatures (Tm) are matched. In addition, if possible, amplicons are kept at 100 bp or shorter and probes at 20 bp or shorter. Following the successful identification of primer and probe sets, specificity of the primers is assessed using Primer-BLAST (Ye et al., 2012).

Custom primer and probe mixes (primer to probe ratio of 3.6) are ordered from IDT. By convention, mutant probes are ordered using the FAM dye, whereas wild-type probes are labeled with HEX. In parallel, as a positive control, a gBlock gene fragment (IDT) of the interrogated locus with the mutation of interest is designed. Within the designed amplicon three bases that lie outside the probe sequences are scrambled to act as a potential targeting point to identify gBlock contamination if necessary. Alternatively, a distinct sequence with an already established ddPCR strategy can be included outside the amplicon.

ddPCR Assay

Once received, primer/probe mixes are resuspended according to the manufacturer's protocol to yield a 20× concentrate. GBLOCK™ (gBlock™, IDT) fragments are resuspended in nuclease-free ddH₂O and subsequently diluted to match the gene copies present in the control reaction.

ddPCR is performed on a BioRad platform, using a QX200™ droplet generator, a C1000™ touch cycler, a PX1™ PCR Plate Sealer, and a QX200™ droplet reader.

ddPCR reactions are set up in the following way: 8 μl of DNA solution (30-50 ng of genomic DNA total), 1 μl of the mutant primer/probe mix, 1 μl of the wild type primer/probe mix, 10 μl ddPCR Supermix™ (BioRad, 1863024). Following mixing, the ddPCR reaction and droplet generation oil (BioRad, 1863005) are transferred to a cartridge (BioRad, 1864008) to generate reaction droplets according to the manufacturer's instructions. The emulsified solution is transferred onto a PCR plate (Eppendorf, 951020346) and the PCR protocol is run on the thermocycler (Appendix 2, see below). Following PCR, reactions are analyzed on the droplet reader using QUANTASOFT™ (QuantaSoft™) (BioRad).

For each reaction, at least three independent runs are performed: First, a gradient PCR determines the optimal annealing temperature for each assay using control DNA supplemented with GBLOCK™ (i.e. a PCR template based upon a synthesized oligonucleotide that contains the mutation being assessed) at a 1:10 ratio (number of copies) and non-template controls (NTC). The optimal temperature is defined as the one with the most orthogonal separation along the two axes while allowing maximal distinction of baseline versus signal. Second, a test run employing the chosen temperature is performed on positive controls with the GBLOCK™ at 1:1 and 1:10 ratios, control DNA and NTC. This helps to determine the cutoffs and the false positive rates for the mutation. Third, the experimental run is performed, in which the extracted sperm sample (as a technical triplicate), DNA extracted from control sperm, and NTC will be analyzed. This experiment can be extended by including paternal and maternal samples derived from blood or saliva and patient DNA. Alternatively, these analyses will be performed only in case of detected mosaicism in a later run.

The QUANTASOFT™ (QuantaSoft™) and QUANTASOFT ANALYSIS PRO™ (QuantaSoft Analysis Pro™) software packages (BioRad) allow direct quantification of abundance of mutant versus wild-type allele. The sensitivity of a given assay is variable, however, mosaicism of above 0.1% could be confidently called across all tested conditions. For positive samples, an independent biological replicate is necessary to confirm mosaicism. Risk of transmitting the variant of interest to offspring equals the fractional abundance of the variant in sperm (mutant/[mutant+wild type]).

### Protocol for Design of ‘Haploinsufficiency-Ome’

We have used a number of peer-reviewed publications and public databases to design a haploinsufficiency-ome. Publications quantifying genic mutation intolerance and heterozygous lethality will be considered, along with publications that provide evidence linking heterozygosity in given genes with specific conditions, including ASD, trinucleotide expansion, intellectual disability (ID), congenital heart disease (CHD) and other disorders and diseases. Public databases used include OMIM and SFARI. Through a rigorous screening process that may be partly automated and partly manual, we will optimize our list for genes with high certainty of risk (i.e. odd ratios, increased risk of disease) and magnitude of risk (i.e. severity of disease) following loss of the gene copy.

In Table 1, we show an example of the proposed cross-list screening. The columns are drawn from the following sources:

A: Gene list. Composed of all genes in the lists described in columns D-H, and top tenth of genes ranked by pLI and HI (columns B and C)

B: pLI. Probability of loss-of-function intolerance. Drawn from the ExAC database, see Samocha et al., 2014

C: HI. Probability of haploinsufficiency. List taken from Dataset Si from Huang et al., 2010

D: DDD-monoallelic (Wright et al., 2015)

E: SFARI database gene list (see Iossifov et al., 2014)

F: Autism/ID X-panded Panel Gene List (GeneDx)

G: xGen Inherited Disease Panel (IDT)

H: Lifton/CHD. List of de novo risk genes for congenital heart disease. Taken from Table 2 in Zaidi et al., 2013

### Protocol for Design of Capture Probes From IDT

We have found that the company IDT is the top vendor for oligonucleotides for extraction of various parts of the genome for sequencing. Their strategy is to design 125-mers across areas of the genome that require enrichment. The oligonucleotides are sequenced in 96-well format and are 5′-biotinylated. IDT sells 96 well format of the probes in two different scales: 16 reaction or 96 reaction. Each well contains all of the probes for a single gene, and for each gene the probes, ranging from one to several hundred, are balanced to represent equimolar concentrations. Each reaction contains enough probe for 12 extractions. 96 reactions are enough for 1,000-10,000 individual tests (depending upon the level of optimization). The shelf life for these probes is 3 years if kept frozen. There is an additional modest cost for the ‘blocking oligonucleotides’ which are designed against the ends to prevent false capture.

### Protocol for Generation of Deep Sequencing of ‘Ome’

We will follow standard protocol for exome sequencing, including target capture, streptavidin purification, loading onto a MiSeq™ instrument, which will produce 44-50 million reads per reaction with v3 chemistry. Average of 21 probes per gene, thus for 2000 genes we expect 42000 unique targets. At 1000×42,000,000 we can generate the screening of a single patient samples on a MiSeq™ single run. An alternative method is to barcode the samples from individual patients and multiplex these samples onto a HiSeq™, which offers about 30× more reads per run. On this instrument or on newer generation of Illumina sequencers, cost for data should drop further. Because current exome costs, once fully implemented, we plan to sequence 1/10th of the ‘exome’ at 10× the typical read depth. As an alternative workflow, if capture costs outweigh the cost of deep sequencing, standard ‘whole exome capture kits will be used, and the various ‘ome's will be utilized for post-hoc prioritization of variants.

### Use of ‘Ome’ in a Computational Pipeline

Alignment of the data to the human genome reference will be performed using standard mapping algorithm, currently set by GATK Best Practices, in order to generate a BAM file from each sample. We will plan to perform sequencing of the ‘haploinsufficiency-ome’ at 1000× and saliva at 100× from each male client, to generate two separate sequencing libraries. After mapping of each library using GATK, we will access the programs MuTect™ and Strelka™ in order to identify sperm mosaicism. These two programs are open source, and were determined by head-to-head comparison to be the top performing algorithms to detect mosaicism from about 6 different competing algorithms. The programs output tables that list individual ‘high-confidence’ somatic variants. From these comparisons, our experience is that greater than 20 variants per client will be returned as high confidence by these computer programs. These variants will be ranked based upon ‘damage prediction score’ to identify variants likely to produce loss-of-function. Variants of uncertain effect on protein will not be profiled further. As an alternative workflow, only the sperm sample can be sequenced, skipping the sequencing of saliva from the client, and develop a bioinformatic workflow to identify sperm mosaicism from just the sequencing results. For instance, variants that are identified at near 50% rate can be excluded as non-mosaic, and instead focus analysis on just the small handful of variants that are present below 30% allelic fraction. In alternative embodiments, this could reduce costs, but could lead to more uncertainty about mosaicism.

### Protocol for Generation of ‘Personalized Risk Assessment’ From ‘Ome’.

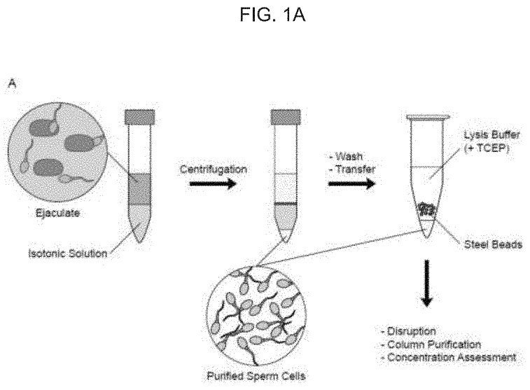

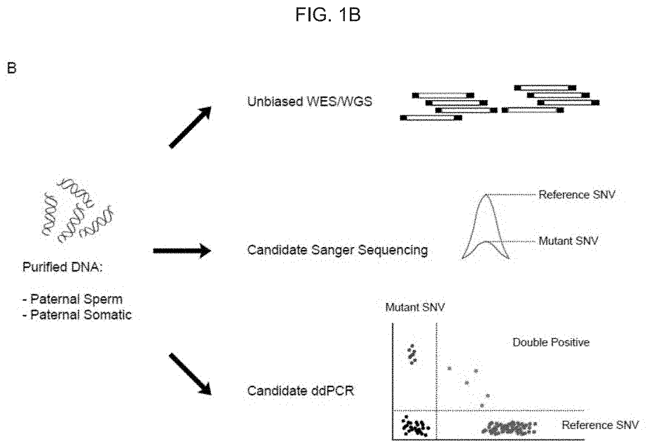

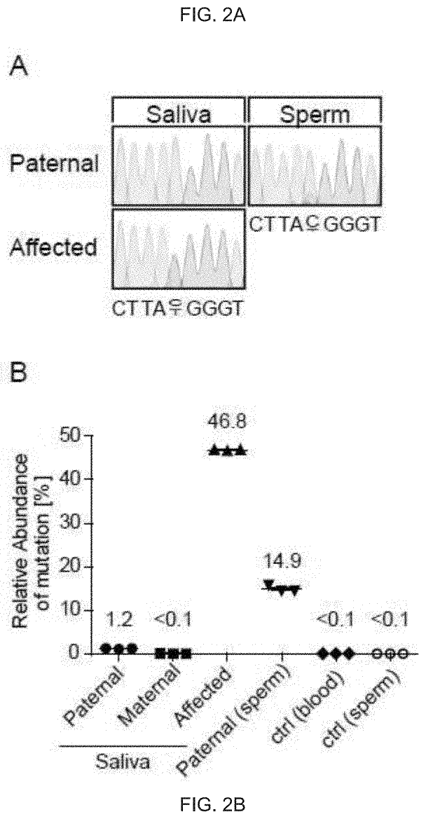

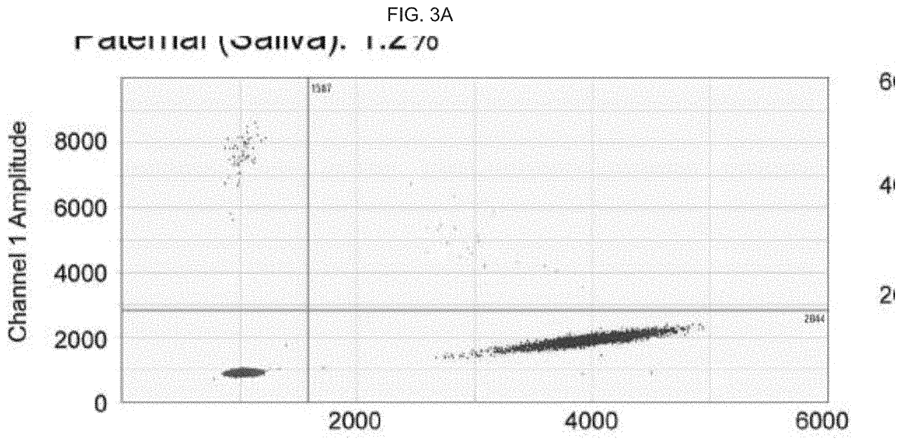

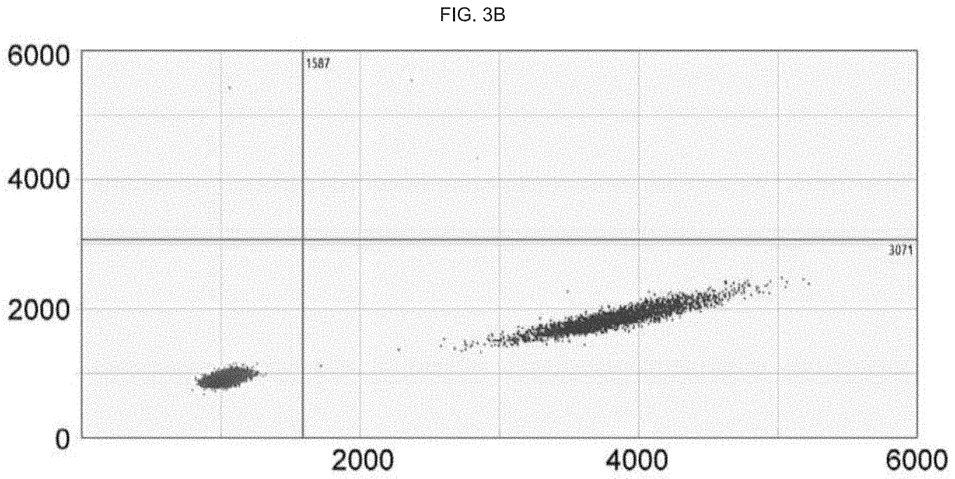

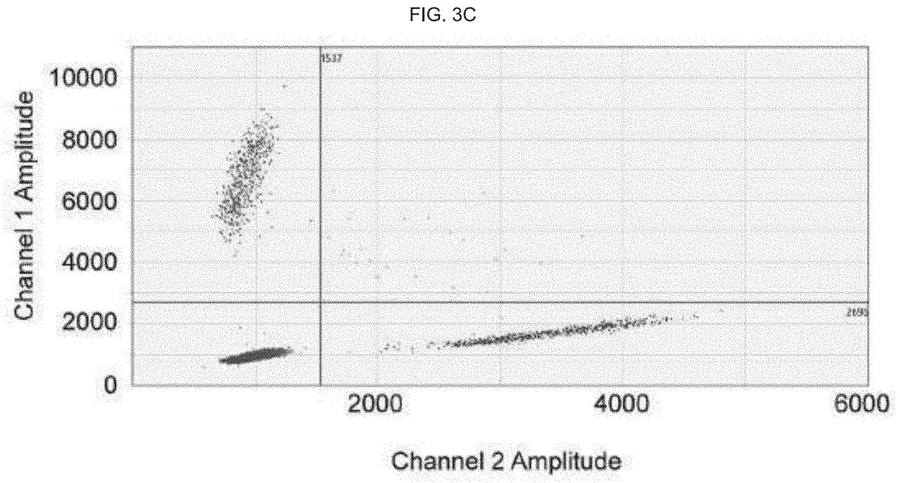

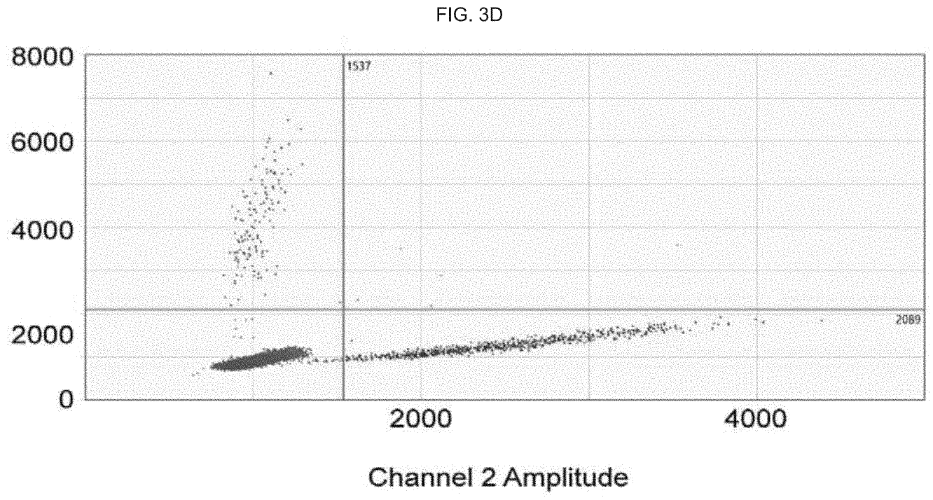

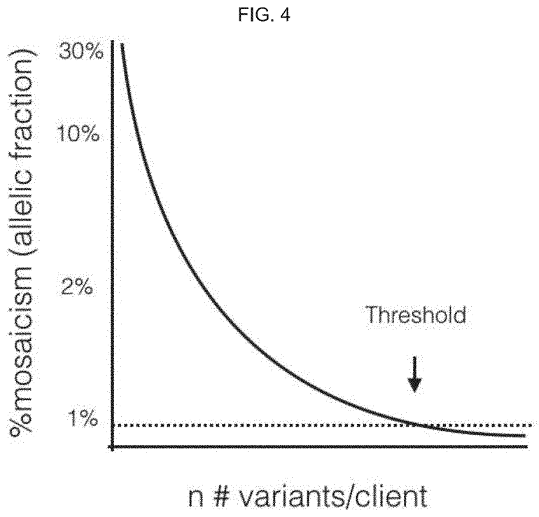

From the sequencing results, the distribution variants are identified with according to the % mosaicism (i.e. allelic fraction, AF, FIG. 4, or FIG. 1, Example 4). FIG. 4 graphically illustrates an example of detection of mosaicism from sperm assessment; a few variants are expected to be detected with % mosaicism above 10%; and there will be an exponential relationship between the number of variants and % mosaicism.

We found just a small number of variants (perhaps just 2-3) that are present with AF>10%. We expect a higher number of variants (perhaps 10-20) that are present with AF between 1-10%. We expect that there will be many variants with AF <1% or that are low-quality variants, and these will be ignored. A report can be generated that highlights either risk below the general population risk, or substantially higher than the general population risk, and will list each gene with mutation above a threshold AF that is part of the ‘haploinsufficiency-ome’, that reaches a specific odds ratio (i.e. above 2). These specific numbers can be modified based upon client preferences. We expect that for most clients, we will be able to establish a risk that is substantially below the baseline risk in the general population of 2-3%, whereas in a small fraction of clients, we will communicate a risk that is substantially above the baseline risk. In this way, individual cases will be stratified according to risk, and the results from this assessment can be directly used in parenting decisions and family planning.

### Appendix 1—Primer3Plus Settings

Primer3Plus File—Do not Edit
Type: Settings
PRIMER_TASK=pick_pcr_primers
PRIMER_PICK_ANYWAY=1
PRIMER_MISPRIMING_LIBRARY=HUMAN
PRIMER_LIB_AMBIGUITY_CODES_CONSENSUS=1
PRIMER_MAX_MISPRIMING=12.00
PRIMER_MAX_TEMPLATE_MISPRIMING=12.00
PRIMER_PAIR_MAX_MISPRIMING=24.00
PRIMER_PAIR_MAX_TEMPLATE_MISPRIMING=24.00
PRIMER_PRODUCT_MIN_TM=
PRIMER_PRODUCT_OPT_TM=
PRIMER_PRODUCT_MAX_TM=
PRIMER_PRODUCT_OPT_SIZE=0
PRIMER_PRODUCT_SIZE_RANGE=40-100 100-150 150-200
PRIMER_GC_CLAMP=0
PRIMER_OPT_SIZE=20
PRIMER_MIN_SIZE=13
PRIMER_MAX_SIZE=27
PRIMER_OPT_TM=55
PRIMER_MIN_TM=54
PRIMER_MAX_TM=56
PRIMER_MAX_DIFF_TM=100.0
PRIMER_MIN_GC=20.0
PRIMER_OPT_GC_PERCENT=
PRIMER_MAX_GC=80.0
PRIMER_SALT_CONC=50.0
PRIMER_DIVALENT_CONC=3.8
PRIMER_DNTP_CONC=0.8
PRIMER_SALT_CORRECTIONS=1
PRIMER_TM_SANTALUCIA=1
PRIMER_DNA_CONC=50.0
PRIMER_NUM_NS_ACCEPTED=0
PRIMER_SELF_ANY=8.00
PRIMER_SELF_END=3.00
PRIMER_MAX_POLY_X=5
PRIMER_LIBERAL_BASE=1
PRIMER_N UM_RETURN=5
PRIMER_FIRST_BASE_INDEX=1
PRIMER_MIN_QUALITY=0
PRIMER_MIN_END_QUALITY=0
PRIMER_QUALITY_RANGE_MIN=0
PRIMER_QUALITY_RANGE_MAX=100
PRIMER_INSIDE_PENALTY=
PRIMER_OUTSIDE_PENALTY=0
PRIMER_MAX_END_STABILITY=9.0
PRIMER_WT_TM_GT=1.0
PRIMER_WT_TM_LT=1.0
PRIMER_WT_SIZE_LT=1.0
PRIMER_WT_SIZE_GT=1.0
PRIMER_WT_GC_PERCENT_LT=0.0
PRIMER_WT_GC_PERCENT_GT=0.0
PRIMER_WT_COMPL_ANY=0.0
PRIMER_WT_COMPL_END=0.0
PRIMER_WT_NUM_NS=0.0
PRIMER_WT_REP_SIM=0.0
PRIMER_WT_SEQ_QUAL=0.0
PRIMER_WT_END_QUAL=0.0
PRIMER_WT_POS_PENALTY=0.0
PRIMER_WT_END_STABILITY=0.0
PRIMER_WT_TEMPLATE_MISPRIMING=0.0
PRIMER_PAIR_WT_PR_PENALTY=1.0
PRIMER_PAIR_WT_IO_PENALTY=0.0
PRIMER_PAIR_WT_DIFF_TM=0.0
PRIMER_PAIR_WT_COMPL_ANY=0.0
PRIMER_PAIR_WT_COMPL_END=0.0
PRIMER_PAIR_WT_PRODUCT_TM_LT=0.0
PRIMER_PAIR_WT_PRODUCT_TM_GT=0.0
PRIMER_PAIR_WT_PRODUCT_SIZE_GT=0.0
PRIMER_PAIR_WT_PRODUCT_SIZE_LT=0.0
PRIMER_PAIR_WT_REP_SIM=0.0
PRIMER_PAIR_WT_TEMPLATE_MISPRIMING=0.0
PRIMER_INTERNAL_OLIGO_OPT_SIZE=
PRIMER_INTERNAL_OLIGO_MIN_SIZE=13
PRIMER_INTERNAL_OLIGO_MAX_SIZE=27
PRIMER_INTERNAL_OLIGO_OPT_TM=56
PRIMER_INTERNAL_OLIGO_MIN_TM=55
PRIMER_INTERNAL_OLIGO_MAX_TM=57.5
PRIMER_INTERNAL_OLIGO_MIN_GC=20.0
PRIMER_INTERNAL_OLIGO_OPT_GC_PERCENT=
PRIMER_INTERNAL_OLIGO_MAX_GC=80.0
PRIMER_INTERNAL_OLIGO_SALT_CONC=50.0
PRIMER_INTERNAL_OLIGO_DIVALENT_CONC=3.8
PRIMER_INTERNAL_OLIGO_DNTP_CONC=0.8
PRIMER_INTERNAL_OLIGO_DNA_CONC=50.0
PRIMER_INTERNAL_OLIGO_SELF_ANY=12.00
PRIMER_INTERNAL_OLIGO_MAX_POLY_X=5
PRIMER_INTERNAL_OLIGO_SELF_END=12.00
PRIMER_INTERNAL_OLIGO_MISHYB_LIBRARY=NONE
PRIMER_INTERNAL_OLIGO_MAX_MISHYB=12.00
PRIMER_INTERNAL_OLIGO_MIN_QUALITY=0
PRIMER_INTERNAL_OLIGO_NUM_NS=0
PRIMER_IO_WT_TM_GT=1.0
PRIMER_IO_WT_TM_LT=1.0
PRIMER_IO_WT_SIZE_LT=1.0
PRIMER_IO_WT_SIZE_GT=1.0
PRIMER_IO_WT_GC_PERCENT_LT=0.0
PRIMER_IO_WT_GC_PERCENT_GT=0.0
PRIMER_IO_WT_COMPL_ANY=0.0
PRIMER_IO_WT_NUM_NS=0.0
PRIMER_IO_WT_REP_SIM=0.0
PRIMER_IO_WT_SEQ_QUAL=0.0
SCRIPT_TASK=Detection
SCRIPT_PRINT_INPUT=0
SCRIPT_FIX_PRIMER_END=5
SCRIPT_CONTAINS_JAVA_SCRIPT=1
SCRIPT_SEQUENCING_LEAD=50
SCRIPT_SEQUENCING_SPACING=500
SCRIPT_SEQUENCING_REVERSE=1
SCRIPT_SEQUENCING_INTERVAL=250
SCRIPT_SEQUENCING_ACCURACY=20
SCRIPT_DETECTION_PICK_LEFT=1
SCRIPT_DETECTION_PICK_HYB_PROBE=1
SCRIPT_DETECTION_PICK_RIGHT=1
SCRIPT_DETECTION_USE_PRODUCT_SIZE=0
SCRIPT_DETECTION_PRODUCT_MIN_SIZE=100
SCRIPT_DETECTION_PRODUCT_OPT_SIZE=200
SCRIPT_DETECTION_PRODUCT_MAX_SIZE=1000
SERVER_PARAMETER_FILE=Default
PRIMER_NAME_ACRONYM_LEFT=F
PRIMER_NAME_ACRONYM_INTERNAL_OLIGO=IN
PRIMER_NAME_ACRONYM_RIGHT=R
PRIMER_NAME_ACRONYM_SPACER=_

### Appendix 2—PCR Protocol

1. 95° C. for 10 minutes
2. 94° C. for 30 seconds
3. X° C. for 1 minute (Temperature depends on assay)

Repeat 2 and 3 for a total of 40 cycles
4. 98° C. for 10 minutes
Steps 2-4 are done with a temperature ramp of 2.0° C./second.

### REFERENCES

1. Dies, K. A. and M. Sahin, Genetics of neurocutaneous disorders: basic principles of inheritance as they apply to neurocutaneous syndromes. Handb Clin Neurol, 2015. 132: p. 3-8.

2. Fromer, M., et al., De novo mutations in schizophrenia implicate synaptic networks. Nature, 2014. 506(7487): p. 179-84.

3. Zaidi, S., et al., De novo mutations in histone-modifying genes in congenital heart disease. Nature, 2013. 498(7453): p. 220-3.

4. Neale, B. M., et al., Patterns and rates of exonic de novo mutations in autism spectrum disorders. Nature, 2012. 485(7397): p. 242-5.

5. Sanders, S. J., et al., De novo mutations revealed by whole-exome sequencing are strongly associated with autism. Nature, 2012. 485(7397): p. 237-41.

6. Bermudez-Lopez, C., et al., Germinal mosaicism in a sample of families with Duchenne/Becker muscular dystrophy with partial deletions in the DMD gene. Genet Test Mol Biomarkers, 2014. 18(2): p. 93-7.

7. Campbell, I. M., et al.,. Parental somatic mosaicism is underrecognized and influences recurrence risk of genomic disorders. Am J Hum Genet, 2012. 95, p. 173-182.

8. Carss, K. J., et al., Exome sequencing improves genetic diagnosis of structural fetal abnormalities revealed by ultrasound. Hum Mol Genet, 2014. 23(12): p. 3269-77.

9. McGrath, J. J., et al., A comprehensive assessment of parental age and psychiatric disorders. JAMA Psychiatry, 2014.71(3): p. 301-9.

10. Pedersen, C. B., et al., The importance of father's age to schizophrenia risk. Mol Psychiatry, 2014. 19(5): p. 530-1.

11. Frans, E. M., et al., Autism risk across generations: a population-based study of advancing grandpaternal and paternal age. JAMA Psychiatry, 2013. 70(5): p. 516-21.

12. Kong, A., et al., Rate of de novo mutations and the importance of father's age to disease risk. Nature, 2012. 488(7412): p. 471-5.

13. Hultman, C. M., et al., Advancing paternal age and risk of autism: new evidence from a population- based study and a meta-analysis of epidemiological studies. Mol Psychiatry, 2011. 16(12): p. 1203-12.

14. Petersen, L., P. B. Mortensen, and C. B. Pedersen, Paternal age at birth of first child and risk of schizophrenia. Am J Psychiatry, 2011. 168(1): p. 82-8.

15. Malaspina, D., et al., Advancing paternal age and the risk of schizophrenia. Arch Gen Psychiatry, 2001. 58(4): p. 361-7.

16. Goriely, A., et al., “Selfish spermatogonia) selection”: a novel mechanism for the association between advanced paternal age and neurodevelopmental disorders. Am J Psychiatry, 2013. 170(6): p. 599-608.

17 Gratten, J., et al., Risk of psychiatric illness from advanced paternal age is not predominantly from de novo mutations. Nat Genet, 2016. 48(7):p. 718-24.

### REFERENCES EXAMPLE 4

Carss, K. J., et al. (2014). Exome sequencing improves genetic diagnosis of structural fetal abnormalities revealed by ultrasound. Hum Mol Genet 23, 3269-3277.

Fromer, M., et al. (2014). De novo mutations in schizophrenia implicate synaptic networks. Nature 506, 179-184.

Homsy, J., et al. (2015). De novo mutations in congenital heart disease with neurodevelopmental and other congenital anomalies. Science 350, 1262-1266.

Huguet, G., Ey, E., and Bourgeron, T. (2013). The genetic landscapes of autism spectrum disorders. Annu Rev Genomics Hum Genet 14, 191-213.

Huang, N., et al. (2010). Characterising and predicting haploinsufficiency in the human genome. PLoS Genet 6, e1001154.

Iossifov, I., et al. (2014). The contribution of de novo coding mutations to autism spectrum disorder. Nature 515, 216-221.

Koressaar, T., and Remm, M. (2007). Enhancements and modifications of primer design program Primer3. Bioinformatics 23, 1289-1291.

Samocha, K. E., et al. (2014). A framework for the interpretation of de novo mutation in human disease. Nat Genet 46, 944-950.

Untergasser, A., Cutcutache, I., Koressaar, T., Ye, J., Faircloth, B. C., Remm, M., and Rozen, S. G. (2012). Primer3—new capabilities and interfaces. Nucleic Acids Res 40, 115.

Untergasser, A., et al. (2007). Primer3Plus, an enhanced web interface to Primer3. Nucleic Acids Res 35, W71-74.

Vissers, L. E., et al. (2015). Rapid method for the isolation of mammalian sperm DNA. Biotechniques 58, 293-300.

Wright, C. F., et al. (2015). Genetic diagnosis of developmental disorders in the DDD study: a scalable analysis of genome-wide research data. Lancet 385, 1305-1314.

Ye, J., et al. (2012). Primer-BLAST: a tool to design target-specific primers for polymerase chain reaction. BMC Bioinformatics 13, 134.

Zaidi, S., et al. (2013). De novo mutations in histone-modifying genes in congenital heart disease. Nature 498, 220-223.

A number of embodiments of the invention have been described. Nevertheless, it can be understood that various modifications may be made without departing from the spirit and scope of the invention. Accordingly, other embodiments are within the scope of the following claims.

1. A method for assessing the genetic makeup of sperm comprising use of a ‘haploinsufficiency-ome’, and optionally using a Digital Droplet PCR (ddPCR) to sequence the genetic makeup of the sperm,
(a) providing a sperm or sperm sample, or sample of the genome of a sperm or sperm sample;
(b) providing a ‘haploinsufficiency-ome’ database, or a compilation of gene sequences, of a comparable species or animal,
wherein providing a ‘haploinsufficiency-ome’ database optionally comprises providing a human ‘haploinsufficiency-ome’ to compare with a human sperm sample,
wherein the ‘haploinsufficiency-ome’ comprises a database or compilation of gene sequences from sperm or haploid precursors thereof;
(c) sequencing the sperm's genome, or the sperm's DNA; and
(d) comparing the sequenced sperm genome or DNA with the ‘haploinsufficiency-ome’ database or compilation of gene sequences, and determining any sequence differences.

2. A method for determining the risk of inheritance of a genetic defect or trait in a younger child or a potential sibling, wherein the younger child or potential sibling has an older sibling having the genetic defect or trait, comprising:
determining the genetic makeup of the sperm of the father of the older sibling using a method of claim 1, and determining whether the genetic makeup of the sperm has the genetic defect or trait found in the older sibling;
wherein determining that the sperm of the father has the genetic defect or trait found in the older sibling indicates a risk that the younger child or the potential sibling will inherit the genetic defect or trait found in the older sibling, or that the genetic defect or trait found in the older sibling will be transmitted to the younger child or the potential sibling.

3. The method of claim 2, wherein the older sibling has autism or autism spectrum disorder (ASD), and a genetic defect or trait found in an ‘autism-ome’ is detected in the sperm of the father, or a specific mutation or allele associated with autism or autism spectrum disorder (ASD) is detected in the sperm of the father, thereby detecting in increased risk of autism or autism spectrum disorder (ASD) in the younger child or the potential sibling.

4. A method for determining the risk of inheritance of a genetic defect or trait, or a haploinsufficient disease or trait, in a younger child or a potential sibling, comprising:
determining the genetic makeup of the sperm of the father using a method of any of the preceding claims, and determining whether the genetic makeup of the sperm comprises a genetic defect or trait, or a haploinsufficient disease or trait, wherein optionally the genetic defect or trait is a de novo genetic defect or trait, and optionally the genetic defect or trait is a genetic defect or trait found in a ‘haploinsufficiency-ome’, or an ‘autism-ome’, or a disease or trait associated with a specific mutation or allele,
wherein determining that the sperm of the father has the genetic defect or trait found indicates a risk that the younger child or the potential sibling will inherit the genetic defect or trait, or that the detected genetic defect or trait will be transmitted to the younger child or the potential sibling,
and optionally the haploinsufficient disease or trait is an autism or autism spectrum disorder (ASD), a trinucleotide expansion, an intellectual disability, a schizophrenia, a heart disease, a congenital heart disease, a neurocutaneous disease, a chromosomal rearrangement, a cancer, dyskeratosis congenita (DKC), Marfan syndrome (MFS) or cleidocranial dysostosis (CCD).

5. A method for determining the risk that a child or potential child has or will have autism or autism spectrum disorder (ASD), comprising:
determining the genetic makeup of the sperm of the father using a method of any of the preceding claims, and determining whether the genetic makeup of the sperm comprises a genetic defect or trait found in an ‘autism-ome’, or a specific mutation or allele associated with the genetic defect or trait, wherein optionally the genetic defect or trait is a de novo genetic defect or trait,
wherein determining that the sperm of the father has the ‘autism-ome’ or specific genetic defect or trait found indicates a risk that the younger child or the potential sibling will inherit autism or autism spectrum disorder (ASD), or that autism or autism spectrum disorder (ASD) will be transmitted to the younger child or the potential sibling.

6. A kit or a product of manufacture comprising components for practicing the method of claim 1.

7. (canceled)

8. A method for determining the presence of a genetic or DNA variation in a sample from an individual,
wherein the genetic or DNA variation comprises: a Structural Variant (SV), a single nucleotide variant (SNV), or an indel (comprising mutations resulting in either insertion or deletion, or both insertion and deletion, of bases in DNA),
the method comprising:
(a)
(i) providing:
a tissue, fluid, blood, serum, sperm or sperm sample, or a sample of the genome of or a genome derived from the tissue, fluid, blood, serum, sperm or sperm sample, or
DNA from or DNA derived from a tissue, fluid, blood, serum, sperm or sperm sample;
(ii) detecting a variation or a mutation in a DNA from (or in) the sample, or detecting a variation or a mutation in the sequence of the DNA from (or in) the sample,
wherein the DNA is analyzed (and the variation or the mutation in the DNA is detected, or the sequence of the DNA is determined) by a method comprising use of:
(1) breakpoint polymerase chain reaction (PCR) to detect a DNA breakpoint comprising use of a set of nested primers that span the junction of a structural variant (SV), wherein optionally the presence of the DNA breakpoint can be detected at frequencies less than (<) 1%;
(2) digital droplet PCR (ddPCR) or an emulsion PCR method to quantify mutations at the level of individual chromosomes;
(3) restriction site mutation (RSM) detection comprising use of a set of nested primers that span a single-nucleotide variant, wherein a mutation can be detected by first eliminating the reference sequence by digestion with a restriction enzyme followed by amplification of the mutant sequence by serial PCR reactions using nested primers;
(4) any combination of (1) and (2), (1) and (3), (2) and (3), or (1), (2) and (3); or
(5) whole genome sequencing; or
(b) detecting a variation or a mutation in a DNA from (or in) a sample, or detecting a variation or a mutation in the sequence of the DNA from (or in) a sample,
wherein the sample comprises
a tissue, fluid, blood, serum, sperm or sperm sample, or a sample of the genome of or a genome derived from the tissue, fluid, blood, serum, sperm or sperm sample, or
DNA from or DNA derived from a tissue, fluid, blood, serum, sperm or sperm sample;
and the DNA is analyzed, or the sequence of the DNA is determined, by a method comprising use of:
(1) breakpoint polymerase chain reaction (PCR) to detect a DNA breakpoint comprising use of a set of nested primers that span the junction of a structural variant (SV), wherein optionally the presence of the DNA breakpoint can be detected at frequencies <1%;
(2) digital droplet PCR (ddPCR) or an emulsion PCR method to quantify mutations at the level of individual chromosomes;
(3) restriction site mutation (RSM) detection comprising use of a set of nested primers that span a single-nucleotide variant, wherein a mutation can be detected by first eliminating the reference sequence by digestion with a restriction enzyme followed by amplification of the mutant sequence by serial PCR reactions using nested primers;
(4) any combination of (1) and (2), (1) and (3), (2) and (3), or (1), (2) and (3); or
(5) whole genome sequencing.

9. The method of claim 8, further comprising quantifying a mutation frequency of the DNA variation or a mutation to provide an estimate of the risk of the presence or possible occurrence of a disease, trait or disorder caused by the genetic mutation or variation in an offspring or a potential future child.

10. The method of claim 8, wherein the method is used as a Non-Invasive Prenatal Test (NIPT) when the father is known to carry a mutation in his sperm and the same mutation is undetectable in the blood of the mother prior to her pregnancy,
wherein detection of the DNA variation or mutation in the mothers blood, serum or plasma, during pregnancy determines the presence or occurrence of the genetic mutation in the fetus, and thereby also provides an estimate of the risk of the presence or possible occurrence of a disease, trait or disorder caused by the genetic mutation or variation in the child or fetus.

11. The method of claim 8, wherein an older sibling has autism or autism spectrum disorder (ASD), and a genetic defect or trait is detected in the DNA of the sperm of the father, or a specific mutation or allele associated with autism or autism spectrum disorder (ASD) is detected in the sperm of the father, thereby detecting an increased risk of autism or autism spectrum disorder (ASD) in the younger child or the potential sibling.

12. The method of claim 8, wherein the disease or disorder is a haploinsufficient or dominant disease or trait.

13. The method of claim 8, wherein the disease, trait or disorder is: an autism or autism spectrum disorder (ASD), a trinucleotide expansion, an intellectual disability, a schizophrenia, a heart disease, a congenital heart disease, a neurocutaneous disease, a chromosomal rearrangement, a cancer, dyskeratosis congenita (DKC), Marfan syndrome (MFS) or cleidocranial dysostosis (CCD).

14. A kit or a product of manufacture comprising components for practicing the method of claim 8, wherein optionally the kit or the product of manufacture comprises PCR primers for detecting a desired genetic defect, disease or trait, and optionally the kit or the product of manufacture comprises instructions for practicing the method of any of the preceding claims.

15. (canceled)

16. The method of claim 1, wherein the sequencing comprises using a method comprising a Digital Droplet polymerase chain reaction (PCR) (ddPCR, digital PCR or dePCR), or equivalent, optionally a QX200™ Droplet Digital™ PCR System (BIO-RAD).

17. The method of claim 1, wherein the ‘haploinsufficiency-ome’ is: a “disease-ome”, or a panel of genes that produce or are associated with haploinsufficient birth defects or other diseases wherein one copy of a gene is defective, mutated or missing; or, a “hereditable condition-ome”, or a panel of genes that produce or are associated with a hereditable condition or trait, wherein one copy of a gene is defective, mutated or missing.

18. The method of claim 1, wherein the “disease-ome” or “hereditable condition-ome” is or comprises:
an ‘autism-ome’, or a panel of genes that produce or are associated with autism, or autism spectrum disorder (ASD), wherein one copy of the gene is defective, mutated or missing, or having a specific mutation or allele associated with autism or ASD,
a ‘schizophrenia-ome’ or a panel of genes that produce or are associated with schizophrenia, wherein one copy of the gene is defective, mutated or missing, or having a specific mutation or allele associated with schizophrenia,
a ‘congenital heart disease-ome’, or a panel of genes that produce or are associated with congenital heart disease, wherein one copy of the gene is defective, mutated or missing, or having a specific mutation or allele associated with congenital heart disease,
a spina bifida-ome or a panel of genes that produce or are associated with spina bifida, wherein one copy of the gene is defective, mutated or missing, or having a specific mutation or allele associated with spina bifida, or
a compilation of gene sequences of any disease class or hereditable condition or trait class where one or more de novo mutations are known to contribute (optionally substantially contributing) to a risk of a child acquiring or inheriting the disease or hereditable condition or trait.

19. The method of claim 1, wherein the genetic makeup of the sperm is screened for the presence of a genetic defect, hereditable condition or trait, wherein a finding or a determination of one or more sequence differences in step (d) in the sperm sample versus the “disease-ome” or “hereditable condition-ome” is a finding or determination that a progeny of the sperm is at risk, optionally at high risk, of developing or inheriting the disease, condition or trait.

20. The method of claim 1, wherein the genetic makeup of the sperm is screened for a de novo genetic mutation, or the genetic defect or trait comprises a de novo genetic mutation, and optionally if the one or more sequence differences in step (d) in the sperm sample versus the “disease-ome” or “hereditable condition-ome” is a finding or determination that the sperm has a de novo genetic mutation, then this is a finding or determination that a progeny of the sperm is at risk, optionally at high risk, of inheriting the de novo genetic mutation.

21. The method of claim 1, wherein the sperm is a human sperm.

22. The method of claim 1, wherein the sperm is a non-human sperm.
